(19) United States
(12) Patent Application Publication
(10) Pub. No.: US 2019/0112571 A1
(43) Pub. Date: Apr. 18, 2019

(54) PROCESSING OF BIOMASS MATERIALS

(71) Applicant: XYLECO, INC., Wakefield, MA (US)

(72) Inventors: Marshall Medoff, Wakefield, MA (US); Thomas Craig Masterman, Rockport, MA (US); Michael W. Finn, Somerville, MA (US)

(21) Appl. No.: 15/031,444

(22) PCT Filed: Mar. 30, 2016
(86) PCT No.: PCT/US2016/024964

§ 371 Date: Apr. 22, 2016

Related U.S. Application Data
(60) Provisional application No. 62/140,793, filed on Mar. 31, 2015.

(51) Int. Cl. C12N 1/06; C12N 1/20; C12N 1/22; C12P 7/56

(52) U.S. Cl. CPC C12N 1/06; C12N 1/20; C12P 2203/00; C12P 7/56; C12N 1/22

(57) ABSTRACT

The use of cell matter in fermentation mixtures for producing a product is disclosed. In embodiments, the product comprises carbohydrates, alcohols, or organic acids (e.g., lactic acid or succinic acid), or mixtures thereof.

### RELATED APPLICATIONS

This application claims priority to U.S. Provisional Application No. 62/140,793, filed on Mar. 31, 2015. The entire contents of this application are hereby incorporated by reference.

### FIELD OF THE INVENTION

The present invention relates generally to methods and compositions comprising the use of lysed cell matter in fermentation processes to produce a product.

### BACKGROUND

As demand for petroleum increases, so too does interest in renewable feedstocks for manufacturing biofuels and biochemicals. One of the most attractive sources of renewable feedstock is lignocellulosic biomass, derived from the fibrous, dry matter of plants. The use of lignocellulosic biomass as a feedstock has been studied since the 1970s and has gained widespread attention due to its renewable nature, abundance, and ability for domestic production. Many potential sources of lignocellulosic biomass are available today, including products and residues from agricultural and forestry sectors. At present, these materials are typically used as animal feed or are composted, burned in a cogeneration facility, or buried in landfills.

Lignocellulosic biomass is comprised of crystalline cellulose fibrils embedded in a hemicellulose matrix, surrounded by lignin. This compact matrix is recalcitrant to degradation by enzymes and other chemical, biochemical and biological processes due to the rigid nature of the plant cell walls. Cellulosic biomass (e.g., biomass material from which substantially all the lignin has been removed) can be more accessible to enzymes and other conversion processes compared with lignocellulosic biomass, but even so, the overall hydrolytic yield of these materials is still remarkably low.

While a number of methods have been explored to extract structural carbohydrates from lignocellulosic biomass, these methods are either are too expensive, produce too low a yield, leave undesirable chemicals in the resulting product, or simply degrade the sugars. Carbohydrates from renewable biomass sources could become the basis of food, biochemicals, and fuels industries by replacing, supplementing, or substituting petroleum and other fossil feedstocks. However, techniques need to be developed that will make these monosaccharides available in large quantities and at acceptable purities and prices. Therefore, there is a considerable need for alternative methods to breakdown lignocellulosic biomass that is high-yielding, inexpensive, and does not destroy the carbohydrate hydrolysis products.

### SUMMARY OF THE INVENTION

Efforts to produce food, biochemicals, and biofuels from renewable feedstocks, such as biomass, require the use of multiple processing steps. These processing steps serve to breakdown the biomass from complex, recalcitrant structures into tractable and desirable materials. In order to streamline production, individual biomass processing steps are often combined or carried out in a single reactor vessel. However, the combination of these biomass processing steps may have a negative impact on various downstream processes, e.g., fermentation, which can be slowed or even inhibited in the presence of byproducts from earlier processes. Provided herein are methods for production of sugars and sugar products derived from the processing of biomass. Specifically, these methods rely on the use of lysed cell matter, e.g., lysed bacterial or fungal cells, in order to enhance the saccharification and/or fermentation steps and reduce the need for addition of expensive nutrients. While not wishing to be bound by theory, the lysed cell matter provides at least the following advantages: a) reduced inhibition of a biological process, such as a fermentation process, resulting from one or more processing steps that occurred prior to the biological process; b) an inexpensive source material for nutrients required for biological processes, such as saccharification and/or fermentation processes and c) improvement of the selectivity of a target product, such as producing a specific stereoisomer, such as D- or L-lactic acid. It is believed that the lysed cells allow for inhibitors to be adsorbed out of solution, due in part, to their high surface area while providing particular nutrients that reduce the stress encountered by an organism when confronted with inhibitors.

In one aspect, the present invention provides a method of making a product, the method comprising contacting one or more sugars with a fermentation composition comprising lysed cell matter to produce the product. In some embodiments, the one or more sugars comprise oligosaccharides, polysaccharides, tetrasaccharides, trisaccharides, disaccharides, monosaccharides, or mixtures of any of these. In some embodiments, the one or more sugars comprise disaccharides and monosaccharides. In some embodiments, the one or more sugars comprise glucose, galactose, mannose, lactose, fructose, maltose, and xylose. In an embodiment, the one or more sugars comprise glucose and xylose.

In some embodiments, the one or more sugars are formed by saccharifying a biomass material comprising cellulosic or lignocellulosic material, such as corn cobs and/or corn stover. In some embodiments, the biomass material comprises lignocellulosic material. In some embodiments, the lignocellulosic material comprises an agricultural product or waste, a paper product or waste, a forestry product or waste, or a general waste. In some embodiments, the agricultural product or waste comprises sugar cane, jute, hemp, flax, bamboo, sisal, alfalfa, hay, arracacha, buckwheat, banana, barley, cassava, kudzu, oca, sago, sorghum, potato, sweet potato, taro, yams, beans, favas, lentils, peas, grasses, grain residues, canola straw, wheat straw, barley straw, oat straw, rice straw, rice bran, silage, abaca, corn, corn cobs, corn stover, corn fiber, corn kernels, corn stalks, soybean stover, alfalfa, hay, coconut hair, cotton seed hair, nut shells, palm fronds, carrot processing waste, molasses spent wash, vegetable oil byproducts, beet pulp, bagasse, rice hulls, oat hulls, barley hulls, wheat chaff, or beeswing. In some embodiments, the agricultural product or waste comprises sugar cane, corn, corn cobs, corn stover, corn fiber, corn kernels, or corn stalks. In some embodiments, the agricultural product or waste comprises corn, corn cobs, corn stover, or corn stalks. In some embodiments, the paper product or waste comprises paper, pigmented papers, loaded papers, coated papers, filled papers, magazines, printed matter, printer paper, polycoated paper, cardstock, cardboard, paperboard, or paper pulp. In some embodiments, the forestry product or waste comprises aspen wood, particle board, wood chips, or sawdust. In some embodiments, the general waste comprises manure, sewage, or offal.

In some embodiments, the lignocellulosic material has been pretreated to reduce its recalcitrance. In some embodiments, the recalcitrance of the lignocellulosic material has been reduced by treating the lignocellulosic material with an electron beam, sonication, oxidation, pyrolysis, steam explosion, heat treatment, chemical treatment, mechanical treatment, or freeze grinding. In some embodiments, the recalcitrance of the lignocellulosic material has been reduced by treating the lignocellulosic material with an electron beam, for example, electrons accelerated through a potential difference of between 0.8 MV and 5 MV, e.g., between 3.5 MV, between 0.85 MV and 3 MV or between about 0.9 MV and about 2 MV, and at a beam current of between about 50 mA and 250 mA, e.g., 75 mA and 200 mA or between about 90 mA and 160 mA.

In some embodiments, the one or more sugars are isolated prior to contact with the fermentation composition, e.g., the solids are separated from the liquids and then the liquids can be purified. In some embodiments, the method of isolation comprises filtration, fractionation, extraction, precipitation, solubilization, chromatography, centrifugation, or other separation technique.

In some embodiments, the lysed cell matter comprises lysed cells from a microorganism. In some embodiments, the microorganism comprises a protist, a protozoan, an algae, a yeast, a fungus, a bacterium, or an archaeon. In some embodiments, the lysed cell matter comprises lysed bacterial or fungal cells. In some embodiments, the cells prior to being lysed are in the form of spheres, stars, rods, spirals, helices, and/or in the form of mycelia.

In some embodiments, the lysed cell matter comprises lysed fungal cells. In some embodiments, the fungal cells comprise a species in the genera selected from Coprinus, Myceliophthora, Scytalidium, Penicillium, Aspergillus, Humicola, Fusarium, Thielavia, Acremonium, Chrysosporium, Saccharomyces, Candida, Clavispora, Pichia, Yarrowia, or Trichoderma. In some embodiments, the fungal cells comprise a species in the genus Trichoderma. In some embodiments, the fungal cells comprise the species Trichoderma reesei. In some embodiments, the Trichoderma reesei comprises any individual strain, variant, or mutant thereof, e.g., Trichoderma reesei QM6a, Trichoderma reesei RL-P37, Trichoderma reesei MCG-80, Trichoderma reesei RUTC30, Trichoderma reesei RUT-NG14, Trichoderma reesei PC3-7, or Trichoderma reesei QM9414. In some embodiments, the Trichoderma reesei comprises Trichoderma reesei strain RUTC30.

In some embodiments, the lysed cell matter is produced by sonication, homogenization, chemical treatment, mechanical treatment, freeze thawing, or other similar techniques, such as centrifugation, heat treatment or osmostic lysis. In other embodiments, combinations of these lysing treatments are used in any order.

In some embodiments, the concentration of lysed cell matter in the fermentation composition is greater than or equal to about 1% by volume, e.g., greater than or equal to about 2%, about 3%, about 4%, or about 5% by volume or more. In some embodiments, the concentration of lysed cell matter is greater than or equal to about 5%, e.g., about 5%, about 10%, about 15%, about 20%, about 25%, about 30%, about 35%, about 40%, about 45%, about 50%, about 55%, about 60%, about 65%, about 70%, about 75%, about 80%, or even greater e.g., about 95% by volume. In some embodiments, the concentration of lysed cell matter in the fermentation composition is greater than or equal to about 10%, e.g., about 10%, about 15%, about 20%, about 25%, about 30%, about 35%, about 40%, about 45%, about 50%, about 55%, about 60%, about 65%, about 70%, or about 75%. In some embodiments, the concentration of lysed cell matter in the fermentation composition is greater than or equal to about 25%, e.g., about 25%, about 30%, about 35%, about 40%, about 45%, about 50%, about 55%, or about 60%. In some embodiments, the concentration of lysed cell matter in the fermentation composition is greater than or equal to about 40%, e.g., about 40%, about 45%, or about 50%.

In some embodiments, the fermentation composition further comprises a fermentation agent. In some embodiments, the fermentation agent comprises one or more living cells. In some embodiments, the fermentation agent comprises a prokaryote. In some embodiments, the prokaryote comprises one or more bacteria, fungi, or archaea. In some embodiments, the prokaryote comprises one or more bacteria. In some embodiments, the one or more bacteria comprise a species in the genera selected from Bacillus, Actinobacillus, Lactobacillus, Leuconostoc, Pediococcus, Lactococcus, Streptococcus, Weisella, or Pseudomonas. In some embodiments, the one or more bacteria comprise a species in the genera selected from Actinobacillus, Lactobacillus, Leuconostoc, or Lactococcus.

In some embodiments, the fermentation composition further comprises an additive. In some embodiments, the additive comprises a surfactant, an antifoaming agent, an antimicrobial agent, a pH adjusting agent (e.g., an acid or a base), a solid support (such as an organic or inorganic solid support), or a processed cell product. In some embodiments, the surfactant comprises an ionic surfactant, a non-ionic surfactant, an amphoteric surfactant, a detergent, or an organic solvent. In some embodiments, the antifoaming agent is an oil, an alcohol, a powder, a polyacrylate, a silicon-based agent, or polyglycol (e.g., polyethylene glycol or polypropylene glycol) or polyether (e.g., antifoam 204) dispersions. In some embodiments, the antimicrobial agent is an antibacterial or antifungal agent. In some embodiments, the pH adjusting agent is an acid (e.g., HCl, AcOH, H₂SO₄, H₃PO₄, citric acid, malic acid, succinic acid, or lactic acid). In some embodiments, the pH adjusting agent is a base (e.g., NaOH, KOH, Ca(OH)₂, or NH₃). In some embodiments, the processed cell product comprises yeast extract, chitin powder, or materials or residue from cell culture.

In some embodiments, the one or more sugars are maintained at a temperature greater than or equal to about 35° C., e.g., about 35° C., about 36° C., about 37° C., about 38° C., about 39° C., about 40° C., about 41° C., about 42° C., about 43° C., about 44° C., about 45° C., about 50° C., about 55° C., or about 60° C. In some embodiments, the one or more sugars are maintained at a temperature greater than or equal to about 35° C., e.g., about 35° C., about 36° C., about 37° C., about 38° C., about 39° C., about 40° C., about 41° C., about 42° C., about 43° C., about 44° C., or about 45° C. In some embodiments, the one or more sugars are maintained at a temperature equal to or greater than about 40° C.

During fermentation, the pH is maintained to sustain the life of the organism and to maximize product formation. For an acid-loving organism, the pH can be maintained between about 2.5 and about 5.5, e.g., between about 3 and about 4.5 or between about 3.4 and about 4.2. For base-loving organisms, the pH can be maintained between about 8 and 10, e.g., between about 8.5 and 9.5 or between about 8.6 and 9.3. For organisms preferring neutral conditions, the pH can be maintained between about 6 and about 8.5 or between about 6.5 and about 8.0 or between about 7.0 and 7.8.

In some embodiments, the duration of the method is between 0 and about 100 hours, e.g., about 5 hours, about 10 hours, about 15 hours, about 20 hours, about 25 hours, about 30 hours, about 35 hours, about 40 hours, about 45 hours, about 50 hours, about 55 hours, about 60 hours, about 65 hours, about 70 hours, about 75 hours, about 80 hours, about 85 hours, about 90 hours, about 95 hours, or about 100 hours. In some embodiments, the duration of the method is between 0 and about 75 hours, e.g., about 5 hours, about 10 hours, about 15 hours, about 20 hours, about 25 hours, about 30 hours, about 35 hours, about 40 hours, about 45 hours, about 50 hours, about 55 hours, about 60 hours, about 65 hours, about 70 hours, or about 75 hours. In some embodiments, the duration of the method is between 0 and about 50 hours, e.g., about 5 hours, about 10 hours, about 15 hours, about 20 hours, about 25 hours, about 30 hours, about 35 hours, about 40 hours, about 45 hours, or about 50 hours.

In some embodiments, the product comprises carbohydrates, alcohols, or organic acids. In some embodiments, the product comprises organic acids. In some embodiments, the organic acids comprise polyhydroxy acids, alpha-hydroxy acids or beta-hydroxy acids. In some embodiments, the organic acids comprise lactic acid, succinic acid, glycolic acid, citric acid, malic acid, or tartaric acid. In some embodiments, the organic acids comprise lactic acid. In some embodiments, the organic acids comprise succinic acid.

In some embodiments, the product comprises a mixture of isomers. In some embodiments, the product comprises a mixture of L- and D-isomers. In some embodiments, the product comprises a mixture of L- and D-isomers of lactic acid. In some embodiments, the product is nearly pure (e.g., about 95%, about 96% or about 97% ee) L-lactic acid or nearly pure (e.g., about 95%, about 96%, or about 97% ee) D-lactic acid.

In some embodiments, the mixture comprises a ratio of L-lactic acid to D-lactic acid greater than or equal to 50:50, e.g., about 55:45, about 60:40, about 65:35, about 70:30, about 75:25, about 80:20, about 85:15, about 90:10, about 95:5, about 99:1 or more. In some embodiments, the mixture comprises a ratio of L-lactic acid to D-lactic acid of about 60:40. In some embodiments, the mixture comprises a ratio of L-lactic acid to D-lactic acid of about 80:20. In some embodiments, the mixture comprises a ratio of L-lactic acid to D-lactic acid of about 90:10. In some embodiments, the mixture comprises a ratio of L-lactic acid to D-lactic acid of about 95:5 or more. In some embodiments, the mixture comprises a ratio of L-lactic acid to D-lactic acid of about 99:1 or more. In some embodiments, the mixture comprises a ratio of L-lactic acid to D-lactic acid less than or equal to 50:50, e.g., about 45:55, about 40:60, about 35:65, about 30:70, about 25:75, about 20:80, about 15:85, about 10:90, about 5:95, about 1:99 or less. In some embodiments, the mixture comprises a ratio of L-lactic acid to D-lactic acid of about 40:60. In some embodiments, the mixture comprises a ratio of L-lactic acid to D-lactic acid of about 20:80. In some embodiments, the mixture comprises a ratio of L-lactic acid to D-lactic acid of about 10:90. In some embodiments, the mixture comprises a ratio of L-lactic acid to D-lactic acid of about 5:95. In some embodiments, the mixture comprises a ratio of L-lactic acid to D-lactic acid of about 1:99 or less.

In some embodiments, the product is further isolated. In some embodiments, the method of isolation comprises precipitation, crystallization, chromatography (e.g., SMB), centrifugation, distillation (e.g., vacuum distillation), or extraction.

In some embodiments, the method is carried out in a fluid medium, e.g., an aqueous solution. In some embodiments, the method is performed in a tank, e.g., a carbon steel, stainless steel, or ceramic-lined tank. In many embodiments, the tank is configured to control the temperature of the contents within, e.g., includes a jacket, e.g., a steam trace, half-pipe or a dimpled jacket.

In some embodiments, the method further comprises contacting a biomass comprising lignocellulosic material with a saccharification composition to produce a saccharified biomass. In some embodiments, the saccharified biomass comprises one or more sugars. In some embodiments, the one or more sugars comprise oligosaccharides, polysaccharides, tetrasaccharides, trisaccharides, disaccharides, monosaccharides, or mixtures of any of these. In some embodiments, the one or more sugars comprise disaccharides and monosaccharides. In some embodiments, the one or more sugars comprise glucose, galactose, mannose, lactose, fructose, maltose, and xylose. In an embodiment, the one or more sugars comprise glucose and xylose.

In some embodiments, the saccharification composition comprises a saccharification agent. In some embodiments, the saccharification agent comprises one or more living cells or a biomass-degrading enzyme. In some embodiments, the one or more living cells comprise fungal cells. In some embodiments, the fungal cells comprise a species from the genera Coprinus, Myceliophthora, Scytalidium, Penicillium, Aspergillus, Humicola, Fusarium, Thielavia, Acremonium, Chrysosporium, Saccharomyces, Candida, Clavispora, Pichia, Yarrowia, or Trichoderma. In some embodiments, the fungal cells comprise a species in the genus Trichoderma. In some embodiments, the fungal cells comprise the species Trichoderma reesei. In some embodiments, the Trichoderma reesei comprises any individual strain, variant, or mutant thereof, e.g., Trichoderma reesei QM6a, Trichoderma reesei RL-P37, Trichoderma reesei MCG-80, Trichoderma reesei RUTC30, Trichoderma reesei RUT-NG14, Trichoderma reesei PC3-7, or Trichoderma reesei QM9414. In some embodiments, the Trichoderma reesei comprises strain RUTC30.

In some embodiments, the biomass-degrading enzyme is derived from fungal cells. In some embodiments, the fungal cells comprise a species from the genera Coprinus, Myceliophthora, Scytalidium, Penicillium, Aspergillus, Humicola, Fusarium, Thielavia, Acremonium, Chrysosporium, Saccharomyces, Candida, Clavispora, Pichia, Yarrowia, or Trichoderma. In some embodiments, the fungal cells comprise a species in the genus Trichoderma. In some embodiments, the fungal cells comprise the species Trichoderma reesei. In some embodiments, the Trichoderma reesei comprises any individual strain, variant, or mutant thereof, e.g., Trichoderma reesei QM6a, Trichoderma reesei RL-P37, Trichoderma reesei MCG-80, Trichoderma reesei RUTC30, Trichoderma reesei RUT-NG14, Trichoderma reesei PC3-7, or Trichoderma reesei QM9414. In some embodiments, the Trichoderma reesei comprises strain RUTC30.

In some embodiments, the biomass-degrading enzyme is an endoglucanase, an exoglucanase, a cellobiase, a cellobiohydrolase, a xylanase, a ligninase, or a hemicellulase. In some embodiments, the biomass-degrading enzyme is an endoglucanase, an exoglucanase, a cellobiase, a cellobiohydrolase, a xylanase, a ligninase, or a hemicellulase derived from a fungal cell. In some embodiments, the biomass-degrading enzyme is an endoglucanase, an exoglucanase, a cellobiase, a cellobiohydrolase, a xylanase, a ligninase, or a hemicellulase derived from a species from the genera Coprinus, Myceliophthora, Scytalidium, Penicillium, Aspergillus, Humicola, Fusarium, Thielavia, Acremonium, Chrysosporium, Saccharomyces, Candida, Clavispora, Pichia, Yarrowia, or Trichoderma. In some embodiments, the biomass-degrading enzyme is an endoglucanase, an exoglucanase, a cellobiase, a cellobiohydrolase, a xylanase, a ligninase, or a hemicellulase derived from Trichoderma, e.g., Trichoderma reesei, e.g., any individual strain, variant, or mutant thereof, e.g., Trichoderma reesei QM6a, Trichoderma reesei RL-P37, Trichoderma reesei MCG-80, Trichoderma reesei RUTC30, Trichoderma reesei RUT-NG14, Trichoderma reesei PC3-7, or Trichoderma reesei QM9414. In some embodiments, the biomass-degrading enzyme is a cellobiase, a cellobiohydrolase, a ligninase, or a hemicellulase derived from Trichoderma reesei or any individual strain, variant, or mutant thereof.

In another aspect, the present invention provides a composition comprising one or more sugars and a fermentation composition comprising lysed cell matter. In some embodiments, the one or more sugars comprise oligosaccharides, polysaccharides, tetrasaccharides, trisaccharides, disaccharides, monosaccharides, or mixtures of these. In some embodiments, the one or more sugars comprise disaccharides and monosaccharides. In some embodiments, the one or more sugars comprise glucose, galactose, mannose, lactose, fructose, maltose, and xylose. In an embodiment, the one or more sugars comprise glucose and xylose.

In some embodiments, the one or more sugars are formed by saccharifying a biomass material comprising cellulosic or lignocellulosic material, such as corn cobs and/or corn stover. In some embodiments, the biomass material comprises lignocellulosic material. In some embodiments, the lignocellulosic material comprises an agricultural product or waste, a paper product or waste, a forestry product or waste, or a general waste. In some embodiments, the agricultural product or waste comprises sugar cane, jute, hemp, flax, bamboo, sisal, alfalfa, hay, arracacha, buckwheat, banana, barley, cassava, kudzu, oca, sago, sorghum, potato, sweet potato, taro, yams, beans, favas, lentils, peas, grasses, grain residues, canola straw, wheat straw, barley straw, oat straw, rice straw, rice bran, silage, abaca, corn, corn cobs, corn stover, corn fiber, corn kernels, corn stalks, soybean stover, alfalfa, hay, coconut hair, cotton seed hair, nut shells, palm fronds, carrot processing waste, molasses spent wash, vegetable oil byproducts, beet pulp, bagasse, rice hulls, oat hulls, barley hulls, wheat chaff, or beeswing. In some embodiments, the agricultural product or waste comprises sugar cane, corn, corn cobs, corn stover, corn fiber, corn kernels, or corn stalks. In some embodiments, the agricultural product or waste comprises corn, corn cobs, corn stover, or corn stalks. In some embodiments, the paper product or waste comprises paper, pigmented papers, loaded papers, coated papers, filled papers, magazines, printed matter, printer paper, polycoated paper, cardstock, cardboard, paperboard, or paper pulp. In some embodiments, the forestry product or waste comprises aspen wood, particle board, wood chips, or sawdust. In some embodiments, the general waste comprises manure, sewage, or offal.

In some embodiments, the lignocellulosic material has been pretreated to reduce its recalcitrance. In some embodiments, the recalcitrance of the lignocellulosic material has been reduced by treating the lignocellulosic material with an electron beam, sonication, oxidation, pyrolysis, steam explosion, heat treatment, chemical treatment, mechanical treatment, or freeze grinding. In some embodiments, the recalcitrance of the lignocellulosic material has been reduced by treating the lignocellulosic material with an electron beam, for example, electrons accelerated through a potential difference of between 0.8 MV and 5 MV, e.g., between 3.5 MV, between 0.85 MV and 3 MV or between about 0.9 MV and about 2 MV, and at a beam current of between about 50 mA and 250 mA, e.g., 75 mA and 200 mA or between about 90 mA and 160 mA.

In some embodiments, the one or more sugars are isolated prior to contact with the fermentation composition, e.g., the solids are separated from the liquids and then the liquids can be purified. In some embodiments, the method of isolation comprises filtration, fractionation, extraction, precipitation, solubilization, chromatography, centrifugation, or other separation technique.

In some embodiments, the lysed cell matter comprises lysed cells from a microorganism. In some embodiments, the microorganism comprises a protist, a protozoan, an algae, a yeast, a fungus, a bacterium, or an archaeon. In some embodiments, the lysed cell matter comprises lysed bacterial or fungal cells. In some embodiments, the cells prior to being lysed are in the form of spheres, stars, rods, spirals, helices, and/or in the form of mycelia.

In some embodiments, the lysed cell matter comprises lysed fungal cells. In some embodiments, the fungal cells comprise a species in the genera selected from Coprinus, Myceliophthora, Scytalidium, Penicillium, Aspergillus, Humicola, Fusarium, Thielavia, Acremonium, Chrysosporium, Saccharomyces, Candida, Clavispora, Pichia, Yarrowia, or Trichoderma. In some embodiments, the fungal cells comprise a species in the genus Trichoderma. In some embodiments, the fungal cells comprise the species Trichoderma reesei. In some embodiments, the Trichoderma reesei comprises any individual strain, variant, or mutant thereof, e.g., Trichoderma reesei QM6a, Trichoderma reesei RL-P37, Trichoderma reesei MCG-80, Trichoderma reesei RUTC30, Trichoderma reesei RUT-NG14, Trichoderma reesei PC3-7, or Trichoderma reesei QM9414. In some embodiments, the Trichoderma reesei comprises strain RUTC30.

In some embodiments, the lysed cell matter is produced by sonication, homogenization, chemical treatment, mechanical treatment, freeze thawing, or other similar techniques, such as centrifugation, heat treatment or osmostic lysis. In other embodiments, combinations of these lysing treatments are used in any order.

In some embodiments, the concentration of lysed cell matter in the fermentation composition is greater than or equal to about 1% by volume, e.g., greater than or equal to about 2%, about 3%, about 4%, or about 5% by volume or more. In some embodiments, the concentration of lysed cell matter is greater than or equal to about 5%, e.g., about 5%, about 10%, about 15%, about 20%, about 25%, about 30%, about 35%, about 40%, about 45%, about 50%, about 55%, about 60%, about 65%, about 70%, about 75%, about 80%, or even greater e.g., about 95% by volume. In some embodiments, the concentration of lysed cell matter in the fermentation composition is greater than or equal to about 10%, e.g., about 10%, about 15%, about 20%, about 25%, about 30%, about 35%, about 40%, about 45%, about 50%, about 55%, about 60%, about 65%, about 70%, or about 75%. In some embodiments, the concentration of lysed cell matter in the fermentation composition is greater than or equal to about 25%, e.g., about 25%, about 30%, about 35%, about 40%, about 45%, about 50%, about 55%, or about 60%. In some embodiments, the concentration of lysed cell matter in the fermentation composition is greater than or equal to about 40%, e.g., about 40%, about 45%, or about 50%.

In some embodiments, the fermentation composition further comprises a fermentation agent. In some embodiments, the fermentation agent comprises one or more living cells. In some embodiments, the fermentation agent comprises a prokaryote. In some embodiments, the prokaryote comprises one or more bacteria, fungi, or archaea. In some embodiments, the prokaryote comprises one or more bacteria. In some embodiments, the one or more bacteria comprise a species in the genera selected from Bacillus, Actinobacillus, Lactobacillus, Leuconostoc, Pediococcus, Lactococcus, Streptococcus, Weisella, or Pseudomonas. In some embodiments, the one or more bacteria comprise a species in the genera selected from Actinobacillus, Lactobacillus, Leuconostoc, or Lactococcus.

In some embodiments, the fermentation composition further comprises an additive. In some embodiments, the additive comprises a surfactant, an antifoaming agent, an antimicrobial agent, a pH adjusting agent (e.g., an acid or a base), a solid support (such as an organic or inorganic solid support), or a processed cell product. In some embodiments, the surfactant comprises an ionic surfactant, a non-ionic surfactant, an amphoteric surfactant, a detergent, or an organic solvent. In some embodiments, the antifoaming agent is an oil, an alcohol, a powder, a polyacrylate, a silicon-based agent, or polyglycol (e.g., polyethylene glycol or polypropylene glycol) or polyether (e.g., antifoam 204) dispersions. In some embodiments, the antimicrobial agent is an antibacterial or antifungal agent. In some embodiments, the pH adjusting agent is an acid (e.g., HCl, AcOH, H₂SO₄, H₃PO₄, citric acid, malic acid, succinic acid, or lactic acid). In some embodiments, the pH adjusting agent is a base (e.g., NaOH, KOH, Ca(OH)₂, NaHCO₃, CaCO₃, or NH₃). In some embodiments, the processed cell product comprises yeast extract, chitin powder, or materials or residue from cell culture.

In some embodiments, the one or more sugars are maintained at a temperature greater than or equal to about 35° C., e.g., about 35° C., about 36° C., about 37° C., about 38° C., about 39° C., about 40° C., about 41° C., about 42° C., about 43° C., about 44° C., about 45° C., about 50° C., about 55° C., or about 60° C. In some embodiments, the one or more sugars are maintained at a temperature greater than or equal to about 35° C., e.g., about 35° C., about 36° C., about 37° C., about 38° C., about 39° C., about 40° C., about 41° C., about 42° C., about 43° C., about 44° C., or about 45° C. In some embodiments, the one or more sugars are maintained at a temperature equal to or greater than about 40° C.

During fermentation, the pH is maintained to sustain the life of the organism and to maximize product formation. For an acid-loving organism, the pH can be maintained between about 2.5 and about 5.5, e.g., between about 3 and about 4.5 or between about 3.4 and about 4.2. For base-loving organisms, the pH can be maintained between about 8 and 10, e.g., between about 8.5 and 9.5 or between about 8.6 and 9.3. For organisms preferring neutral conditions, the pH can be maintained between about 6 and about 8.5 or between about 6.5 and about 8.0 or between about 7.0 and 7.8.

In some embodiments, the duration of the fermentation is between 0 and about 100 hours, e.g., about 5 hours, about 10 hours, about 15 hours, about 20 hours, about 25 hours, about 30 hours, about 35 hours, about 40 hours, about 45 hours, about 50 hours, about 55 hours, about 60 hours, about 65 hours, about 70 hours, about 75 hours, about 80 hours, about 85 hours, about 90 hours, about 95 hours, or about 100 hours. In some embodiments, the duration of the fermentation is between 0 and about 75 hours, e.g., about 5 hours, about 10 hours, about 15 hours, about 20 hours, about 25 hours, about 30 hours, about 35 hours, about 40 hours, about 45 hours, about 50 hours, about 55 hours, about 60 hours, about 65 hours, about 70 hours, or about 75 hours. In some embodiments, the duration of the method is between 0 and about 50 hours, e.g., about 5 hours, about 10 hours, about 15 hours, about 20 hours, about 25 hours, about 30 hours, about 35 hours, about 40 hours, about 45 hours, or about 50 hours.

In some embodiments, the composition comprises carbohydrates, alcohols, or organic acids. In some embodiments, the composition comprises organic acids. In some embodiments, the organic acids comprise polyhydroxy acids, alpha-hydroxy acids or beta-hydroxy acids. In some embodiments, the organic acids comprise lactic acid, succinic acid, glycolic acid, citric acid, malic acid, or tartaric acid. In some embodiments, the organic acids comprise lactic acid.

In some embodiments, the composition comprises a mixture of isomers. In some embodiments, the composition comprises a mixture of L- and D-isomers. In some embodiments, the composition comprises a mixture that is nearly pure, e.g., about 95%, about 96% or about 97% ee L-lactic acid or nearly pure, e.g., about 95%, about 96% or about 97% ee D-lactic acid. In some embodiments, the mixture comprises a ratio of L-lactic acid to D-lactic acid greater than or equal to 50:50, e.g., about 55:45, about 60:40, about 65:35, about 70:30, about 75:25, about 80:20, about 85:15, about 90:10, about 95:5. In some embodiments, the mixture comprises a ratio of L-lactic acid to D-lactic acid of at least 60:40. In some embodiments, the mixture comprises a ratio of L-lactic acid to D-lactic acid of at least 80:20. In some embodiments, the mixture comprises a ratio of L-lactic acid to D-lactic acid of at least 90:10. In some embodiments, the mixture comprises a ratio of L-lactic acid to D-lactic acid of at least 95:5.

In some embodiments, the composition is further isolated. In some embodiments, the method of isolation comprises precipitation, crystallization, chromatography (e.g., SMB), centrifugation, distillation (e.g., vacuum distillation), or extraction.

In some embodiments, the fermentation is carried out in a fluid medium, e.g., an aqueous solution. In some embodiments, the fermentation is performed in a tank, e.g., a carbon steel, stainless steel, or ceramic-lined tank. In many embodiments, the tank is configured to control the temperature of the contents within, e.g., includes a jacket, e.g., a steam trace, half-pipe or a dimpled jacket.

In another aspect, the present invention provides a composition comprising a saccharified biomass. In some embodiments, the saccharified biomass comprises one or more sugars. In some embodiments, the one or more sugars comprise oligosaccharides, polysaccharides, tetrasaccharides, trisaccharides, disaccharides, monosaccharides, or mixtures of these. In some embodiments, the one or more sugars comprise disaccharides and monosaccharides. In some embodiments, the one or more sugars comprise glucose, galactose, mannose, lactose, fructose, maltose, and xylose. In an embodiment, the one or more sugars comprise glucose and xylose.

In some embodiments, the one or more sugars are formed by saccharifying a biomass material comprising cellulosic or lignocellulosic material, such as corn cobs and/or corn stover. In some embodiments, the biomass material comprises lignocellulosic material. In some embodiments, the lignocellulosic material comprises an agricultural product or waste, a paper product or waste, a forestry product or waste, or a general waste. In some embodiments, the agricultural product or waste comprises sugar cane, jute, hemp, flax, bamboo, sisal, alfalfa, hay, arracacha, buckwheat, banana, barley, cassava, kudzu, oca, sago, sorghum, potato, sweet potato, taro, yams, beans, favas, lentils, peas, grasses, grain residues, canola straw, wheat straw, barley straw, oat straw, rice straw, rice bran, silage, abaca, corn, corn cobs, corn stover, corn fiber, corn kernels, corn stalks, soybean stover, alfalfa, hay, coconut hair, cotton seed hair, nut shells, palm fronds, carrot processing waste, molasses spent wash, vegetable oil byproducts, beet pulp, bagasse, rice hulls, oat hulls, barley hulls, wheat chaff, or beeswing. In some embodiments, the agricultural product or waste comprises sugar cane, corn, corn cobs, corn stover, corn fiber, corn kernels, or corn stalks. In some embodiments, the agricultural product or waste comprises corn, corn cobs, corn stover, or corn stalks. In some embodiments, the paper product or waste comprises paper, pigmented papers, loaded papers, coated papers, filled papers, magazines, printed matter, printer paper, polycoated paper, cardstock, cardboard, paperboard, or paper pulp. In some embodiments, the forestry product or waste comprises aspen wood, particle board, wood chips, or sawdust. In some embodiments, the general waste comprises manure, sewage, or offal.

In some embodiments, the lignocellulosic material has been pretreated to reduce its recalcitrance. In some embodiments, the recalcitrance of the lignocellulosic material has been reduced by treating the lignocellulosic material with an electron beam, sonication, oxidation, pyrolysis, steam explosion, heat treatment, chemical treatment, mechanical treatment, or freeze grinding. In some embodiments, the recalcitrance of the lignocellulosic material has been reduced by treating the lignocellulosic material with an electron beam, for example, electrons accelerated through a potential difference of between 0.8 MV and 5 MV, e.g., between 3.5 MV, between 0.85 MV and 3 MV or between about 0.9 MV and about 2 MV, and at a beam current of between about 50 mA and 250 mA, e.g., 75 mA and 200 mA or between about 90 mA and 160 mA.

In some embodiments, the composition further comprises a saccharification agent. In some embodiments, the saccharification agent comprises one or more living cells or a biomass-degrading enzyme. In some embodiments, the one or more living cells comprise fungal cells. In some embodiments, the fungal cells comprise a species from the genera Coprinus, Myceliophthora, Scytalidium, Penicillium, Aspergillus, Humicola, Fusarium, Thielavia, Acremonium, Chrysosporium, Saccharomyces, Candida, Clavispora, Pichia, Yarrowia, or Trichoderma. In some embodiments, the fungal cells comprise a species in the genus Trichoderma. In some embodiments, the fungal cells comprise the species Trichoderma reesei. In some embodiments, the Trichoderma reesei comprises any individual strain, variant, or mutant thereof, e.g., Trichoderma reesei QM6a, Trichoderma reesei RL-P37, Trichoderma reesei MCG-80, Trichoderma reesei RUTC30, Trichoderma reesei RUT-NG14, Trichoderma reesei PC3-7, or Trichoderma reesei QM9414. In some embodiments, the Trichoderma reesei comprises strain RUTC30.

In some embodiments, the biomass-degrading enzyme is derived from fungal cells. In some embodiments, the fungal cells comprise a species from the genera Coprinus, Myceliophthora, Scytalidium, Penicillium, Aspergillus, Humicola, Fusarium, Thielavia, Acremonium, Chrysosporium, Saccharomyces, Candida, Clavispora, Pichia, Yarrowia, or Trichoderma. In some embodiments, the fungal cells comprise a species in the genus Trichoderma. In some embodiments, the fungal cells comprise the species Trichoderma reesei. In some embodiments, the Trichoderma reesei comprises any individual strain, variant, or mutant thereof, e.g., Trichoderma reesei QM6a, Trichoderma reesei RL-P37, Trichoderma reesei MCG-80, Trichoderma reesei RUTC30, Trichoderma reesei RUT-NG14, Trichoderma reesei PC3-7, or Trichoderma reesei QM9414. In some embodiments, the Trichoderma reesei comprises strain RUTC30.

In some embodiments, the biomass-degrading enzyme is an endoglucanase, an exoglucanase, a cellobiase, a cellobiohydrolase, a xylanase, a ligninase, or a hemicellulase. In some embodiments, the biomass-degrading enzyme is an endoglucanase, an exoglucanase, a cellobiase, a cellobiohydrolase, a xylanase, a ligninase, or a hemicellulase derived from a fungal cell. In some embodiments, the biomass-degrading enzyme is an endoglucanase, an exoglucanase, a cellobiase, a cellobiohydrolase, a xylanase, a ligninase, or a hemicellulase derived from a species from the genera Coprinus, Myceliophthora, Scytalidium, Penicillium, Aspergillus, Humicola, Fusarium, Thielavia, Acremonium, Chrysosporium, Saccharomyces, Candida, Clavispora, Pichia, Yarrowia, or Trichoderma. In some embodiments, the biomass-degrading enzyme is an endoglucanase, an exoglucanase, a cellobiase, a cellobiohydrolase, a xylanase, a ligninase, or a hemicellulase derived from Trichoderma, e.g., Trichoderma reesei, e.g., any individual strain, variant, or mutant thereof, e.g., Trichoderma reesei QM6a, Trichoderma reesei RL-P37, Trichoderma reesei MCG-80, Trichoderma reesei RUTC30, Trichoderma reesei RUT-NG14, Trichoderma reesei PC3-7, or Trichoderma reesei QM9414. In some embodiments, the biomass-degrading enzyme is a cellobiase, a cellobiohydrolase, a ligninase, or a hemicellulase derived from Trichoderma reesei or any individual strain, variant, or mutant thereof.

In some embodiments, the composition further comprises an additive. In some embodiments, the additive comprises a surfactant, an antifoaming agent, an antimicrobial agent, a pH adjusting agent (e.g., an acid or a base), a solid support (such as an organic or inorganic solid support), or a processed cell product. In some embodiments, the surfactant comprises an ionic surfactant, a non-ionic surfactant, an amphoteric surfactant, a detergent, or an organic solvent. In some embodiments, the antifoaming agent is an oil, an alcohol, a powder, a polyacrylate, a silicon-based agent, or polyglycol (e.g., polyethylene glycol or polypropylene glycol) or polyether (e.g., antifoam 204) dispersions. In some embodiments, the antimicrobial agent is an antibacterial or antifungal agent. In some embodiments, the pH adjusting agent is an acid (e.g., HCl, AcOH, H₂SO₄, H₃PO₄, citric acid, malic acid, succinic acid, or lactic acid). In some embodiments, the pH adjusting agent is a base (e.g., NaOH, KOH, Ca(OH)₂, NaHCO₃, CaCO₃, or NH₃). In some embodiments, the processed cell product comprises yeast extract, chitin powder, or materials or residue from cell culture.

In another aspect, the present invention provides a composition comprising a product produced by contacting one or more sugars with a fermentation composition comprising lysed cell matter. In some embodiments, the product comprises carbohydrates, alcohols, or organic acids. In some embodiments, the product comprises organic acids. In some embodiments, the organic acids comprise polyhydroxy acids, alpha-hydroxy acids or beta-hydroxy acids. In some embodiments, the organic acids comprise lactic acid, succinic acid, glycolic acid, citric acid, malic acid, or tartaric acid. In some embodiments, the organic acids comprise lactic acid.

In some embodiments, the one or more sugars comprise oligosaccharides, polysaccharides, tetrasaccharides, trisaccharides, disaccharides, monosaccharides, or mixtures of any of these. In some embodiments, the one or more sugars comprise disaccharides and monosaccharides. In some embodiments, the one or more sugars comprise glucose, galactose, mannose, lactose, fructose, maltose, and xylose. In an embodiment, the one or more sugars comprise glucose and xylose.

In some embodiments, the one or more sugars are formed by saccharifying a biomass material comprising cellulosic or lignocellulosic material, such as corn cobs and/or corn stover. In some embodiments, the biomass material comprises lignocellulosic material. In some embodiments, the lignocellulosic material comprises an agricultural product or waste, a paper product or waste, a forestry product or waste, or a general waste. In some embodiments, the agricultural product or waste comprises sugar cane, jute, hemp, flax, bamboo, sisal, alfalfa, hay, arracacha, buckwheat, banana, barley, cassava, kudzu, oca, sago, sorghum, potato, sweet potato, taro, yams, beans, favas, lentils, peas, grasses, grain residues, canola straw, wheat straw, barley straw, oat straw, rice straw, rice bran, silage, abaca, corn, corn cobs, corn stover, corn fiber, corn kernels, corn stalks, soybean stover, alfalfa, hay, coconut hair, cotton seed hair, nut shells, palm fronds, carrot processing waste, molasses spent wash, vegetable oil byproducts, beet pulp, bagasse, rice hulls, oat hulls, barley hulls, wheat chaff, or beeswing. In some embodiments, the agricultural product or waste comprises sugar cane, corn, corn cobs, corn stover, corn fiber, corn kernels, or corn stalks. In some embodiments, the agricultural product or waste comprises corn, corn cobs, corn stover, or corn stalks. In some embodiments, the paper product or waste comprises paper, pigmented papers, loaded papers, coated papers, filled papers, magazines, printed matter, printer paper, polycoated paper, cardstock, cardboard, paperboard, or paper pulp. In some embodiments, the forestry product or waste comprises aspen wood, particle board, wood chips, or sawdust. In some embodiments, the general waste comprises manure, sewage, or offal.

In some embodiments, the lignocellulosic material has been pretreated to reduce its recalcitrance. In some embodiments, the recalcitrance of the lignocellulosic material has been reduced by treating the lignocellulosic material with an electron beam, sonication, oxidation, pyrolysis, steam explosion, heat treatment, chemical treatment, mechanical treatment, or freeze grinding. In some embodiments, the recalcitrance of the lignocellulosic material has been reduced by treating the lignocellulosic material with an electron beam, for example, electrons accelerated through a potential difference of between 0.8 MV and 5 MV, e.g., between 3.5 MV, between 0.85 MV and 3 MV or between about 0.9 MV and about 2 MV, and at a beam current of between about 50 mA and 250 mA, e.g., 75 mA and 200 mA or between about 90 mA and 160 mA.

In some embodiments, the one or more sugars are isolated prior to contact with the fermentation composition, e.g., the solids are separated from the liquids and then the liquids can be purified. In some embodiments, the method of isolation comprises filtration, fractionation, extraction, precipitation, solubilization, chromatography, centrifugation, or other separation technique.

In some embodiments, the lysed cell matter comprises lysed cells from a microorganism. In some embodiments, the microorganism comprises a protist, a protozoan, an algae, a yeast, a fungus, a bacterium, or an archaeon. In some embodiments, the lysed cell matter comprises lysed bacterial or fungal cells. In some embodiments, the cells prior to being lysed are in the form of spheres, stars, rods, spirals, helices, and/or in the form of mycelia.

In some embodiments, the lysed cell matter comprises lysed fungal cells. In some embodiments, the fungal cells comprise a species in the genera selected from Coprinus, Myceliophthora, Scytalidium, Penicillium, Aspergillus, Humicola, Fusarium, Thielavia, Acremonium, Chrysosporium, Saccharomyces, Candida, Clavispora, Pichia, Yarrowia, or Trichoderma. In some embodiments, the fungal cells comprise a species in the genus Trichoderma. In some embodiments, the fungal cells comprise the species Trichoderma reesei. In some embodiments, the Trichoderma reesei comprises any individual strain, variant, or mutant thereof, e.g., Trichoderma reesei QM6a, Trichoderma reesei RL-P37, Trichoderma reesei MCG-80, Trichoderma reesei RUTC30, Trichoderma reesei RUT-NG14, Trichoderma reesei PC3-7, or Trichoderma reesei QM9414. In some embodiments, the Trichoderma reesei comprises Trichoderma reesei strain RUTC30.

In some embodiments, the lysed cell matter is produced by sonication, homogenization, chemical treatment, mechanical treatment, freeze thawing, or other similar techniques, such as centrifugation, heat treatment or osmostic lysis. In other embodiments, combinations of these lysing treatments are used in any order.

In some embodiments, the concentration of lysed cell matter in the fermentation composition is greater than or equal to about 1% by volume, e.g., greater than or equal to about 2%, about 3%, about 4%, or about 5% by volume or more. In some embodiments, the concentration of lysed cell matter is greater than or equal to about 5%, e.g., about 5%, about 10%, about 15%, about 20%, about 25%, about 30%, about 35%, about 40%, about 45%, about 50%, about 55%, about 60%, about 65%, about 70%, about 75%, about 80%, or even greater e.g., about 95% by volume. In some embodiments, the concentration of lysed cell matter in the fermentation composition is greater than or equal to about 10%, e.g., about 10%, about 15%, about 20%, about 25%, about 30%, about 35%, about 40%, about 45%, about 50%, about 55%, about 60%, about 65%, about 70%, or about 75%. In some embodiments, the concentration of lysed cell matter in the fermentation composition is greater than or equal to about 25%, e.g., about 25%, about 30%, about 35%, about 40%, about 45%, about 50%, about 55%, or about 60%. In some embodiments, the concentration of lysed cell matter in the fermentation composition is greater than or equal to about 40%, e.g., about 40%, about 45%, or about 50%.

In some embodiments, the fermentation composition further comprises a fermentation agent. In some embodiments, the fermentation agent comprises one or more living cells. In some embodiments, the fermentation agent comprises a prokaryote. In some embodiments, the prokaryote comprises one or more bacteria, fungi, or archaea. In some embodiments, the prokaryote comprises one or more bacteria. In some embodiments, the one or more bacteria comprise a species in the genera selected from Bacillus, Actinobacillus, Lactobacillus, Leuconostoc, Pediococcus, Lactococcus, Streptococcus, Weisella, or Pseudomonas. In some embodiments, the one or more bacteria comprise a species in the genera selected from Actinobacillus, Lactobacillus, Leuconostoc, or Lactococcus.

In some embodiments, the fermentation composition further comprises an additive. In some embodiments, the additive comprises a surfactant, an antifoaming agent, an antimicrobial agent, a pH adjusting agent (e.g., an acid or a base), a solid support (such as an organic or inorganic solid support), or a processed cell product. In some embodiments, the surfactant comprises an ionic surfactant, a non-ionic surfactant, an amphoteric surfactant, a detergent, or an organic solvent. In some embodiments, the antifoaming agent is an oil, an alcohol, a powder, a polyacrylate, a silicon-based agent, or polyglycol (e.g., polyethylene glycol or polypropylene glycol) or polyether (e.g., antifoam 204) dispersions. In some embodiments, the antimicrobial agent is an antibacterial or antifungal agent. In some embodiments, the pH adjusting agent is an acid (e.g., HCl, AcOH, H₂SO₄, H₃PO₄, citric acid, malic acid, succinic acid, or lactic acid). In some embodiments, the pH adjusting agent is a base (e.g., NaOH, KOH, Ca(OH)₂, NaHCO₃, CaCO₃, or NH₃). In some embodiments, the processed cell product comprises yeast extract, chitin powder, or materials or residue from cell culture.

In some embodiments, the one or more sugars are maintained at a temperature greater than or equal to about 35° C., e.g., about 35° C., about 36° C., about 37° C., about 38° C., about 39° C., about 40° C., about 41° C., about 42° C., about 43° C., about 44° C., about 45° C., about 50° C., about 55° C., or about 60° C. In some embodiments, the one or more sugars are maintained at a temperature greater than or equal to about 35° C., e.g., about 35° C., about 36° C., about 37° C., about 38° C., about 39° C., about 40° C., about 41° C., about 42° C., about 43° C., about 44° C., or about 45° C. In some embodiments, the one or more sugars are maintained at a temperature equal to or greater than about 40° C.

During fermentation, the pH is maintained to sustain the life of the organism and to maximize product formation. For an acid-loving organism, the pH can be maintained between about 2.5 and about 5.5, e.g., between about 3 and about 4.5 or between about 3.4 and about 4.2. For base-loving organisms, the pH can be maintained between about 8 and 10, e.g., between about 8.5 and 9.5 or between about 8.6 and 9.3. For organisms preferring neutral conditions, the pH can be maintained between about 6 and about 8.5 or between about 6.5 and about 8.0 or between about 7.0 and 7.8.

In some embodiments, the duration of the method is between 0 and about 100 hours, e.g., about 5 hours, about 10 hours, about 15 hours, about 20 hours, about 25 hours, about 30 hours, about 35 hours, about 40 hours, about 45 hours, about 50 hours, about 55 hours, about 60 hours, about 65 hours, about 70 hours, about 75 hours, about 80 hours, about 85 hours, about 90 hours, about 95 hours, or about 100 hours. In some embodiments, the duration of the method is between 0 and about 75 hours, e.g., about 5 hours, about 10 hours, about 15 hours, about 20 hours, about 25 hours, about 30 hours, about 35 hours, about 40 hours, about 45 hours, about 50 hours, about 55 hours, about 60 hours, about 65 hours, about 70 hours, or about 75 hours. In some embodiments, the duration of the method is between 0 and about 50 hours, e.g., about 5 hours, about 10 hours, about 15 hours, about 20 hours, about 25 hours, about 30 hours, about 35 hours, about 40 hours, about 45 hours, or about 50 hours.

In some embodiments, the product comprises a mixture of isomers. In some embodiments, the product comprises a mixture of L- and D-isomers. In some embodiments, the product comprises a mixture of L- and D-isomers of lactic acid. In some embodiments, the product comprises nearly pure (e.g., about 95%, about 96% or about 97% ee) L-lactic acid or nearly pure (e.g., about 95%, about 96%, or about 97% ee) D-lactic acid.

In some embodiments, the mixture comprises a ratio of L-lactic acid to D-lactic acid greater than or equal to 50:50, e.g., about 55:45, about 60:40, about 65:35, about 70:30, about 75:25, about 80:20, about 85:15, about 90:10, about 95:5, about 99:1 or more. In some embodiments, the mixture comprises a ratio of L-lactic acid to D-lactic acid of about 60:40. In some embodiments, the mixture comprises a ratio of L-lactic acid to D-lactic acid of about 80:20. In some embodiments, the mixture comprises a ratio of L-lactic acid to D-lactic acid of about 90:10. In some embodiments, the mixture comprises a ratio of L-lactic acid to D-lactic acid of about 95:5. In some embodiments, the mixture comprises a ratio of L-lactic acid to D-lactic acid of at least 99:1 or more. In some embodiments, the mixture comprises a ratio of L-lactic acid to D-lactic acid less than or equal to 50:50, e.g., about 45:55, about 40:60, about 35:65, about 30:70, about 25:75, about 20:80, about 15:85, about 10:90, about 5:95, about 1:99 or less. In some embodiments, the mixture comprises a ratio of L-lactic acid to D-lactic acid of at least 40:60. In some embodiments, the mixture comprises a ratio of L-lactic acid to D-lactic acid of at least 20:80. In some embodiments, the mixture comprises a ratio of L-lactic acid to D-lactic acid of at least 10:90. In some embodiments, the mixture comprises a ratio of L-lactic acid to D-lactic acid of at least 5:95. In some embodiments, the mixture comprises a ratio of L-lactic acid to D-lactic acid of at least 1:99 or less.

In some embodiments, the product produced is further is isolated. In some embodiments, the method of isolation comprises precipitation, crystallization, chromatography (e.g., SMB), centrifugation, distillation (e.g., vacuum distillation), or extraction.

In some embodiments, the fermentation is carried out in a fluid medium, e.g., an aqueous solution. In some embodiments, the fermentation is performed in a tank, e.g., a carbon steel, stainless steel, or ceramic-lined tank. In many embodiments, the tank is configured to control the temperature of the contents within, e.g., includes a jacket, e.g., a steam trace, half-pipe or a dimpled jacket.

In some embodiments, the product is further produced by contacting a biomass comprising lignocellulosic material with a saccharification composition to produce a saccharified biomass. In some embodiments, the saccharified biomass comprises one or more sugars. In some embodiments, the one or more sugars comprise oligosaccharides, polysaccharides, tetrasaccharides, trisaccharides, disaccharides, and monosaccharides, or mixtures of any of these. In some embodiments, the one or more sugars comprise disaccharides and monosaccharides. In some embodiments, the one or more sugars comprise glucose, galactose, mannose, lactose, fructose, maltose, and xylose. In an embodiment, the one or more sugars comprise glucose and xylose.

In some embodiments, the saccharification composition comprises a saccharification agent. In some embodiments, the saccharification agent comprises one or more living cells or a biomass-degrading enzyme. In some embodiments, the one or more living cells comprise fungal cells. In some embodiments, the fungal cells comprise a species from the genera Coprinus, Myceliophthora, Scytalidium, Penicillium, Aspergillus, Humicola, Fusarium, Thielavia, Acremonium, Chrysosporium, Candida, Clavispora, Yarrowia, or Trichoderma. In some embodiments, the fungal cells comprise a species in the genus Trichoderma. In some embodiments, the fungal cells comprise the species Trichoderma reesei. In some embodiments, the Trichoderma reesei comprises any individual strain, variant, or mutant thereof, e.g., Trichoderma reesei QM6a, Trichoderma reesei RL-P37, Trichoderma reesei MCG-80, Trichoderma reesei RUTC30, Trichoderma reesei RUT-NG14, Trichoderma reesei PC3-7, or Trichoderma reesei QM9414. In some embodiments, the Trichoderma reesei comprises strain RUTC30.

In some embodiments, the biomass-degrading enzyme is derived from fungal cells. In some embodiments, the fungal cells comprise a species from the genera Coprinus, Myceliophthora, Scytalidium, Penicillium, Aspergillus, Humicola, Fusarium, Thielavia, Acremonium, Chrysosporium, Candida, Clavispora, Yarrowia, or Trichoderma. In some embodiments, the fungal cells comprise a species in the genus Trichoderma. In some embodiments, the fungal cells comprise the species Trichoderma reesei. In some embodiments, the Trichoderma reesei comprises any individual strain, variant, or mutant thereof, e.g., Trichoderma reesei QM6a, Trichoderma reesei RL-P37, Trichoderma reesei MCG-80, Trichoderma reesei RUTC30, Trichoderma reesei RUT-NG14, Trichoderma reesei PC3-7, or Trichoderma reesei QM9414. In some embodiments, the Trichoderma reesei comprises strain RUTC30.

In some embodiments, the biomass-degrading enzyme is an endoglucanase, an exoglucanase, a cellobiase, a cellobiohydrolase, a xylanase, a ligninase, or a hemicellulase. In some embodiments, the biomass-degrading enzyme is an endoglucanase, an exoglucanase, a cellobiase, a cellobiohydrolase, a xylanase, a ligninase, or a hemicellulase derived from a fungal cell. In some embodiments, the biomass-degrading enzyme is an endoglucanase, an exoglucanase, a cellobiase, a cellobiohydrolase, a xylanase, a ligninase, or a hemicellulase derived from a species from the genera Coprinus, Myceliophthora, Scytalidium, Penicillium, Aspergillus, Humicola, Fusarium, Thielavia, Acremonium, Chrysosporium, Candida, Clavispora, Yarrowia, or Trichoderma. In some embodiments, the biomass-degrading enzyme is an endoglucanase, an exoglucanase, a cellobiase, a cellobiohydrolase, a xylanase, a ligninase, or a hemicellulase derived from Trichoderma, e.g., Trichoderma reesei, e.g., any individual strain, variant, or mutant thereof, e.g., Trichoderma reesei QM6a, Trichoderma reesei RL-P37, Trichoderma reesei MCG-80, Trichoderma reesei RUTC30, Trichoderma reesei RUT-NG14, Trichoderma reesei PC3-7, or Trichoderma reesei QM9414. In some embodiments, the biomass-degrading enzyme is a cellobiase, a cellobiohydrolase, a ligninase, or a hemicellulase derived from Trichoderma reesei or any individual strain, variant, or mutant thereof.

### DETAILED DESCRIPTION

### Definitions

Unless defined otherwise, all technical and scientific terms used herein have the same meaning as commonly understood by one of ordinary skill in the art to which the invention pertains. Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

The terms “a” and “an” refer to one or to more than one (i.e., to at least one) of the grammatical object of the article. By way of example, “a cell” means one cell or more than one cell.

The term “alcohol”, as used herein, refers to a compound containing a hydroxyl group, e.g., —OH group. Representative alcohols include methanol, ethanol, propanol, butanol, isobutanol, or sugar alcohols (e.g., xylitol, erythritol).

The term “biomass”, as used herein, refers to any non-fossilized organic matter. Biomass can be a starchy material, e.g., comprising cellulosic, hemicellulosic, or lignocellulosic material. For example, the biomass can be an agricultural product, a paper product, forestry product, or any intermediate, byproduct, residue or waste thereof, or a general waste. The biomass may be a combination of such materials. In an embodiment, the biomass is processed, e.g., by a saccharification and/or a fermentation reaction described herein, to produce products such as sugars, alcohols, organic acids, or biofuels.

The term “biomass-degrading enzyme”, as used herein, refers to an enzyme that breaks down components of the biomass matter described herein into intermediates or final products. For example, a biomass-degrading enzyme includes at least cellulases, hemicellulases, ligninases, endoglucancases, cellobiases, xylanases, and cellobiohydrolases. Biomass-degrading enzymes are produced by a wide variety of microorganisms, and can be isolated from said microorganisms, such as T. reesei. The biomass degrading enzyme can be endogenously or heterologously expressed.

The terms “sugar”, “carbohydrate”, and “saccharide” are used herein interchangeably and refer to a compound comprising at least carbon, hydrogen, and oxygen atoms. Sugars may also comprise atoms in addition to carbon, hydrogen, and oxygen, and may exist in either the cyclized or open chain forms. Sugars, carbohydrates, or saccharides may be comprised of one unit or more than one unit, e.g., monosaccharide, disaccharide, trisaccharide, or oligosaccharide, or an associated sugar alcohol. The sugars can exist in any stereoisomeric form. The sugars include 2, 3, 4, 5, 6, or more e.g., 7, 8 or more, e.g., 9-16 carbon atoms. Exemplary sugars include erythose, ribose, ribulose, arabinose, glucose, fructose, mannose, galactose, sedoheptulose, sucrose, maltose, lactose, and cellobiose. In some embodiments, the composition includes xylose and glucose. In other embodiments, the compositions include xylose and glucose, along with other saccharides, such as galactose, sucrose, arabinose, mannose, fructose and oligomeric saccharides, such as di-, tri-, tetra-, penta- and hexasaccharides.

The term “fermentation”, as used herein, refers to a process by which a material is metabolized by a microorganism. Fermentation includes the methods and products that are disclosed in U.S. Pat. No. 8,900,841 and U.S. Patent Publication Nos. 2014-0004570 and 2014/0004574, the full disclosures of which are incorporated by reference herein.

The term “lysed cell matter”, as used herein, refers to material derived from cells that have been lysed or ruptured by a number of methods known in the art, e.g., sonication blending, homogenization, chemical treatment, mechanical treatment, freeze thawing, centrifugation, heat treatment, osmotic lysis, enzymatic lysis, and the like. In some embodiments, combinations of any of these lysing treatments may be used in any order.

The term “organic acid”, as used herein, refers to a compound containing an acidic group, e.g., a carboxylic acid group. Organic acids are comprised of at least carbon, hydrogen, and oxygen atoms, and may be further grouped into classes such as polyhydroxy acids, alpha-hydroxy acids, or beta-hydroxy acids. Representative organic acids of the present invention include, e.g., lactic acid, succinic acid, and glycolic acid.

The terms “saccharification”, as used herein, refers to the conversion of material e.g., biomass material (e.g., lignocellulosic biomass material) into its simpler building block components, comprising carbohydrates, alcohols, and/or organic acids.

Other than in the examples herein, or unless otherwise expressly specified, all of the numerical ranges, amounts, values and percentages, such as those for amounts of materials, elemental contents, times and temperatures of reaction, ratios of amounts, and others, in the following portion of the specification and attached claims may be read as if prefaced by the word “about” even though the term “about” may not expressly appear with the value, amount, or range. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible.

### Methods and Compositions

Provided herein are methods of using lysed cell matter as an ingredient in the fermentation of biomass (e.g., pretreated biomass, saccharified biomass). As disclosed herein, biomass (e.g., pretreated biomass, saccharified biomass) is contacted with a fermentation composition comprising lysed cell matter. In some embodiments, the fermentation composition further comprises a fermentation agent (e.g., one or more living cells, e.g., one or more bacteria), and other components (e.g., additives) to convert the treated biomass to useful intermediates and products.

Described herein is a method of converting biomass (e.g., pretreated biomass, saccharified biomass) to a product. The method may include: a) pretreatment of biomass to produce pretreated biomass, b) saccharification of the pretreated biomass to produce saccharified biomass, c) bioprocessing, e.g., fermentation of the saccharified biomass with a bioprocessing, e.g., fermentation composition comprising lysed cell matter, thereby converting the biomass to a product.

### Preparation of Lysed Cell Matter

The methods and compositions described herein involve conversion of a biomass to a product. This conversion process involves contacting a biomass (e.g., pretreated biomass, saccharified biomass, (e.g., one or more sugars) with a fermentation composition comprising lysed cell matter to produce a product. The lysed cell matter described in the present disclosure is obtained by growing cells in conjunction with practices routinely used in the art. The source cell line can be obtained from wild sources, commercial sources, or research organizations, such as ATCC. In some embodiments, the cells are rehydrated and propagated on appropriate media. Representative cell lines comprise species from the genera Bacillus, Actinobacillus, Coprinus, Myceliophthora, Cephalosporium, Scytalidium, Penicillium, Aspergillus, Humicola, Fusarium, Meripilus, Thielavia, Acremonium, Chrysosporium, Clostridium, Saccharomyces, Candida, Clavispora, Erwinia, Ruminococcus, Cellvibrio, Prevotella, Geobacillus, Fibrobacter, Aeromonas, Cellulomonas, Thermoascus, Thermotoga, Chaetomium, Dictyoglomus, Nonomuraea, Paecilomyces, Thermomyces, Pichia, Yarrowia, Streptomyces, Schizophyllum, and Trichoderma. In some embodiments, the cell line is selected from a species in the genera comprising Bacillus (e.g., Bacillus agaradhaerens AC13, Bacillus circulans, Bacillus subtilis subsp. subtilis str. 168, alkalophilic Bacillus (see, e.g., U.S. Pat. No. 3,844,890 and EP Pub. No. 0 458 162)), Coprinus (e.g., Coprinus cinereus), Myceliophthora (e.g., Myceliophthora thermophila, e.g., Myceliophthora thermophila CBS 117.65), Cephalosporium (e.g., Cephalosporium sp. RYM-202, Cephalosporium sp. CBS 535.71), Scytalidium (e.g., Scytalidium thermophilum, see, e.g., U.S. Pat. No. 4,435,307), Penicillium (e.g., Penicillium simplicissimum BT2246), Aspergillus (see, e.g., EP Publication No. 0458162, Aspergillus kawachii, Aspergillus niger), Humicola (e.g., Humicola insolens DSM 1800), Fusarium (e.g., Fusarium oxysporum, (e.g., Fusarium oxysporum DSM 2672)), Meripilus (e.g., Meripilus giganteus), Thielavia (e.g., Thielavia terrestris), Acremonium (e.g., Acremonium sp. CBS 478.94, Acremonium sp. CBS 265.95, Acremonium persicinum CBS 169.65, Acremonium acremonium AHU 9519, Acremonium brachypenium CBS 866.73, Acremonium dichromosporum CBS 683.73, Acremonium obclavatum CBS 311.74, Acremonium pinkertoniae CBS 157.70, Acremonium roseogrisea CBS 134.56, Acremonium incoloratum CBS 146.62, Acremonium furatum CBS 299.70H), Chrysosporium (e.g., Chrysosporium lucknowense), Clostridium (e.g., Clostridium thermocellum NCIB 10682, Clostridium cellulovorans), Erwinia (e.g., Erwinia (Pectobacterium) chrysanthemi D1, Erwinia (Pectobacterium) chrysanthemi SR120A), Ruminococcus (e.g., Ruminococcus albus SY3), Cellvibrio (e.g., Cellvibrio japonicus, Cellvibrio mixtus), Prevotella (e.g., Prevotella (Bacteroides) ruminicola 23), Geobacillus (e.g., Geobacillus stearothermophilus T-6), Fibrobacter (e.g., Fibrobacter succinogenes S85), Aeromonas (e.g., Aeromonas punctata (caviae) ME-1), Cellulomonas (e.g., Cellulomonas fimi), Thermoascus (e.g., Thermoascus aurantiacus), Thermotoga (e.g., Thermotoga maritima), Chaetomium (e.g., Chaetomium thermophilum), Dictyoglomus (e.g., Dictyoglomus thermophilum Rt46B.1), Nonomuraea (e.g., Nonomuraea flexuosa), Paecilomyces (e.g., Paecilomyces variotii Bainier), Thermomyces (e.g., Thermomyces lanuginosus), Streptomyces (e.g., Streptomyces lividans, Streptomyces halstedii JM8, Streptomyces olivaceoviridis E-86, Streptomyces sp. S38, (see, e.g., EP Publication No. 0458162)), Schizophyllum (e.g., Schizophyllum commune), and Trichoderma (e.g., Trichoderma harzianum E58, Trichoderma viride, Trichoderma koningii, Trichoderma reesei, (e.g., Trichoderma reesei QM6a, Trichoderma reesei RL-P37, Trichoderma reesei MCG-80, Trichoderma reesei RUTC30, Trichoderma reesei RUT-NG14, Trichoderma reesei PC3-7, or Trichoderma reesei QM9414), or any variants or mutants thereof. In some embodiments, any one of these species or combinations of species may be used to produce the lysed cell matter.

In some embodiments, the growth of the cell cultures from which the lysed cell matter is derived is conducted with agitation. In some cases, agitation may be performed using jet mixing as described in U.S. Pat. No. 8,636,402, U.S. Pat. No. 8,669,099, and U.S. Patent Publication No. 2012-0091035, the full disclosures of which are incorporated by reference herein.

After growth, the cells are isolated following standard procedures, e.g., centrifugation, ultrafiltration, or other separation techniques. Growth of these cells may yield enzymes (e.g., biomass-degrading enzymes) that can be used in other steps of the process (e.g., saccharification). Enzymes utilized in downstream processes are produced, isolated, and prepared in accordance with methods disclosed in U.S. Publication No. US 2014-0011258 filed Sep. 3, 2013, the full disclosure of which is incorporated herein by reference.

Lysis of the cell matter is carried out after growth and isolation of the cells. In some embodiments, lysis of the cell matter may be accomplished by methods known in the art e.g., sonication, pressure (e.g., cell bomb using pressure differences), blending, homogenization, ball mill agitation, high shear mixing, e.g., pumping the cell matter through a pipe with static mixers, centrifugation (e.g., ultracentrifugation or disk stack centrifugation), heat treatment, mechanical treatment, chemical treatment, freeze thawing, osmotic lysis, or enzymatic lysis.

In some embodiments, only a portion of the cells are lysed, e.g., less than 2%, less than 3%, less than 5%, less than 8%, less than 9%, less than 12%, less than 15%, less than 20%, less than 25%, less than 30%, less than 35%, less than 40%, or less than 50% of the cells are lysed. In other embodiments, nearly are the cells are lysed, e.g., greater than 75%, greater than 80%, greater than 90%, greater than 95%, or greater than 99% of the cells are lysed.

In some embodiments, the lysis is carried out one time. In other embodiments, the lysis is repeated more than one time, e.g., 2 times, 3 times, 4 times, 5 times, 6 times, 7 times, 8 times, 9 times, 10 times, 15 times, or 20 times.

The lysed cell matter as a component in the fermentation composition has at least two advantages with an optional third advantage: a) the lysed cell matter is used in lieu of additional materials that provide nutrients for the fermentation; b) the lysed cell matter acts to reduce inhibition of fermentation process by residual material from earlier processing steps; and optionally, c) the lysed cell matter may increase the selectivity of the fermentation to the most desired product, for example, enhance enantioselectivity.

Often fermentation or other bioprocessing requires the addition of nutrients (e.g., as yeast extract) to provide the necessary components and/or nutrients for the fermentation process. In the present invention, use of lysed cell matter can replace the need to supplement the fermentation reaction with additional nutrients. In many bioprocessing methods, the manufacture of proteins (e.g., enzymes, e.g., biomass-degrading enzymes) is necessary for the production of the desired final products. One byproduct of the protein manufacture step is cell matter, which is used in the present invention, as a nutrient source in the fermentation step. In some embodiments, the lysed cell matter contains all necessary nutrients for the fermentation process. In some embodiments, the lysed cell matter does not contain all of the key requirements for the fermentation process. In these cases, additional minerals will need to be added.

In some embodiments, the lysed cell matter can reduce the effect of inhibition of the fermentation system. In some embodiments, the presence of lysed cell matter can reduce an induction period for the fermentation system. The mechanisms by which the lysed cell matter reduces the inhibition effects or induction period of fermentation is currently unknown. Without being bound by any theory, it may be that the lysed cell matter absorbs or degrades a small molecule or protein which inhibits or poisons the enzyme process.

An important goal of many bioproces sing methods is selectivity toward a desired product, especially in cases wherein companion byproducts exist that are not useful and/or difficult to separate from the desired product. In some embodiments, the lysed cell matter provides selectivity enhancements in the resulting products of the fermentation reaction, including enhancing the enantiomeric ratio within the product mixture. For example, lactic acid in its L- or D-form is a desirable product for processing to polylactic acid. A lactic acid process and a polylactic acid process have been described in U.S. Publication Nos. US PCT/US2014/35467 and US PCT/US2014/35467, both filed Apr. 25, 2014, the full disclosures of which are incorporated herein by reference. In some embodiments, the lysed cell matter provides unusual selectivity improvements. In some embodiments, the selectivity improvements include achievement of an L:D ratio of greater than 10, optionally greater than 15, alternatively greater than 20 for products that have at least one carbon with a chiral center (e.g., lactic acid, glycolic acid, succinic acid).

In some embodiments, the amount of the lysed cell matter required for nutrition, overcoming inhibition, and improved product selectivity is from about 0.05 weight percent of the lysed cell matter up to about 300 weight percent of the lysed cell matter based on the total fermentable sugar. In some embodiments, the amount of lysed cell matter is from about 0.1 to about 200 weight percent based on the total fermentable sugar. In some embodiments, the amount of lysed cell matter is from about 0.25 to about 125 weight percent based on the total fermentable sugar. The lower limit of the amount of lysed cell matter is related to the amount of nutrients required for the fermentation. In some embodiments, the lysed cell matter may be supplemented by materials and additives known in the art for providing nutrients to fermentation processes. When other nutrient sources are used, these supplemental materials and additives can be added in a weight ratio of lysed cell matter:other nutrient source(s) of about 0.1:1 up to about 4: 1. In some embodiments, the weight ratio range of lysed cell matter:other nutrient source(s) is about 0.25:1 to about 2.5:1. In some embodiments, the weight ratio range of lysed cell matter:other nutrient source(s) is about 0.5:1 to about 2:1.

In some embodiments, the lysed cell matter is isolated from the total cell matter used in related bioprocessing steps by e.g., centrifugation, ultrafiltration or other isolation technique. In some embodiments, the amount of cell matter isolated is at least about 2 weight percent based on the entire reactor contents of the cell growth process. In some embodiments, the maximum amount of cell matter isolated is about 50 weight percent. In some embodiments, the amount of isolated cell matter is in a range from about 5 weight percent to about 35 weight percent. In some embodiments, the amount of isolated cell matter is from about 10 weight percent to about 25 weight percent.

### Fermentation or Other Bioprocessing Methods

The present invention provides methods of using lysed cell matter as an ingredient in the fermentation of biomass (e.g., pretreated biomass, saccharified biomass). As disclosed herein, biomass (e.g., pretreated biomass, saccharified biomass (e.g., one or more sugars)) is contacted with a fermentation composition comprising lysed cell matter. In some embodiments, the fermentation composition further comprises a fermentation agent (e.g., one or more living cells, e.g., one or more bacteria), and other components (e.g., additives) to convert the treated biomass to useful intermediates and products.

In some embodiments, the fermentation is carried out for a duration of about 0 to about 200 hours. In some embodiments, the fermentation is carried out for a duration of about 24 to about 168 hours, e.g., about 24 to about 96 hours. In some embodiments, the optimum pH for fermentation is in the range from about pH 4 to about pH 8. In some embodiments, the optimum pH for fermentation is in the range from about pH 4.5 to about pH 8, e.g., about pH 4.5 to about pH 7.5, about pH 5 to about pH 7, about pH 5.5 to about pH 7, about pH 6.0 to about pH 7, about pH 6.5 to about pH 7. In some embodiments, the pH range is dependent on the fermentation agent (e.g., one or more living cells, e.g., one or more bacteria). For instance, the optimum pH for some fungal species (e.g., yeast) is in the range from about pH 4.5 to about pH 5.5, while the optimum pH for some bacterial species is in the range from about pH 4.5 to about pH 7.5. In some embodiments, fermentation is carried out at temperatures in the range of 20° C. to 40° C. (e.g., 26° C. to 40° C.), however thermophilic microorganisms prefer higher temperatures (e.g., greater than or equal to 40° C.).

In some embodiments, e.g., when anaerobic organisms are used, at least a portion of the fermentation is conducted in the absence of oxygen, e.g., under a blanket of an inert gas such as N₂, Ar, He, CO₂ or mixtures thereof. Additionally, the mixture may have a constant purge of an inert gas flowing through the tank during part of or all of the fermentation. In some cases, anaerobic conditions can be achieved or maintained by carbon dioxide production during the fermentation and no additional inert gas is needed.

In some embodiments, all or a portion of the fermentation process can be interrupted before the low molecular weight sugar is completely converted to a product (e.g., an organic acid or alcohol). In these cases, the intermediate fermentation products include carbohydrates (e.g., polysaccharides, oligosaccharides, trisaccharides, disaccharides, monosaccharides, and the like) in high concentrations. In some embodiments, the carbohydrates can be isolated via any means known in the art. In some embodiments, these intermediate fermentation products can be used in preparation of food for human or animal consumption. In some embodiments, the intermediate fermentation products can be ground to a fine particle size in a stainless-steel laboratory mill to produce a flour-like substance.

In some embodiments, the fermentation may be subjected to jet mixing. In some embodiments, the fermentation step is performed in the same reactor (e.g., tank) and earlier steps in the bioproces sing method (e.g., saccharification). Mobile fermenters can be utilized, as described in International App. No. PCT/US2007/074028 (which was filed Jul. 20, 2007, was published in English as WO 2008/011598 and designated the United States), the contents of which is incorporated herein in its entirety. Similarly, the saccharification equipment can be mobile. Further, saccharification and/or fermentation may be performed in part or entirely during transit.

### Fermentation Agents or Other Bioprocessing Agents

The present invention described herein provides methods and compositions wherein lysed cell matter is used as an ingredient in the fermentation of biomass (e.g., pretreated biomass, saccharified biomass). As provided herein, biomass (e.g., pretreated biomass, saccharified biomass (e.g., one or more sugars)) is contacted with a fermentation composition comprising lysed cell matter. In some embodiments, the fermentation composition further comprises a fermentation agent (e.g., one or more living cells, e.g., one or more bacteria), and other components (e.g., additives) to convert the treated biomass to useful intermediates and products.

In some embodiments, the fermentation agent comprises one or more living cells. In some embodiments, the one or more living cells may be a bacterium (including, but not limited to, e.g., a cellulolytic bacterium), a fungus, (including, but not limited to, e.g., a yeast), a plant, a protist, e.g., a protozoa or a fungus-like protest (including, but not limited to, e.g., a slime mold), or an alga. In some embodiments, the one or more living cells comprise a prokaryote. Suitable fermenting cells have the ability to convert carbohydrates, such as glucose, fructose, xylose, arabinose, mannose, galactose, oligosaccharides or polysaccharides into fermentation products. Fermenting cells include strains of the genus Saccharomyces spp. (including, but not limited to, S. cerevisiae (baker's yeast), S. dietetics, S. uvarum), the genus Kluyveromyces, (including, but not limited to, K. marxianus, K. fragilis), the genus Candida (including, but not limited to, C. pseudotropicalis, and C. brassicae), Pichia stipitis (a relative of Candida shehatae), the genus Clavispora (including, but not limited to, C. lusitaniae and C. opuntiae), the genus Pachysolen (including, but not limited to, P. tannophilus), the genus Bretannomyces (including, but not limited to, e.g., B. clausenii (Philippidis, G. P., 1996, Cellulose Bioconversion Technology, in Handbook on Bioethanol: Production and Utilization, Wyman, C. E., ed., Taylor & Francis, Washington, D.C., 179-212)). Other suitable cells for fermentation include, for example, Actinobacillus (e.g., Actinobacillus succinogens), Zymomonas mobilis, Clostridium spp. (including, but not limited to, C. thermocellum (Philippidis, 1996, supra), C. saccharobutylacetonicum, C. saccharobutylicum, C. Puniceum, C. beijernckii, and C. acetobutylicum), Moniliella pollinis, Moniliella megachiliensis, Lactobacillus (e.g., Lactobacillus casei, Lactobacillus rhamnosus, Lactobacillus delbrueckii (e.g., Lactobacillus delbrueckii subspecies delbrueckii, Lactobacillus delbrueckii subspecies lactis), Lactobacillus plantarum, Lactobacillus coryniformis (e.g., Lactobacillus coryniformis subspecies torquens), Lactobacillus pentosus, Lactobacillus brevis), Leuconostoc sp, Pediococcus sp Lactococcus sp Streptococcus sp Weisella sp Pseudomonas sp Yarrowia lipolytica, Aureobasidium sp Trichosporonoides sp Trigonopsis variabilis, Trichosporon sp, Moniliellaacetoabutans sp Typhula variabilis, Candida magnoliae, Ustilaginomycetes sp, Pseudozyma tsukubaensis, Zygosaccharomyces sp., Debaryomyces sp., Hansenula sp., Pichia sp., and Torula sp. In some embodiments, the fermentation agent comprises one or more bacteria. In some embodiments, the one or more bacteria comprise a species in the genera selected from Bacillus, Actinobacillus, Lactobacillus, Leuconostoc, Pediococcus, Lactococcus, Streptococcus, Weisella, or Pseudomonas. In some embodiments, the one or more bacteria comprise a species in the genera selected from Actinobacillus, Lactobacillus, Leuconostoc, or Lactococcus. When the organisms are compatible, mixtures of organisms can be utilized.

For instance, Clostridium spp. can be used in the fermentation process to produce products (e.g., alcohols (ethanol, butanol)), organic acids (e.g., butyric acid, acetic acid), and other organic products (e.g., acetone). In other embodiments, Lactobacillus spp. can be used to produce products (e.g., organic acids (e.g., lactic acid)). In still other embodiments, Actinobacillus succinogens can produce products (e.g., organic acids (e.g., succinic acid)).

In some embodiments, cells that can be used to saccharify biomass material and produce sugars can also be used to ferment and convert those sugars to useful products.

Many such cells and microbial strains are publicly available, either commercially or through research organizations and depositories including the ATCC (American Type Culture Collection, Manassas, Va., USA), the NRRL (Agricultural Research Service Culture Collection, Peoria, Ill., USA), or the DSMZ (Deutsche Sammlung von Mikroorganismen and Zellkulturen GmbH, Braunschweig, Germany), to name a few.

Commercially available yeasts include, for example, Red Star®/Lesaffre Ethanol Red (available from Red Star/Lesaffre, USA), FALI® (available from Fleischmann's Yeast, a division of Burns Philip Food Inc., USA), SUPERSTART® (available from Alltech, now Lallemand), GERT STRAND® (available from Gert Strand AB, Sweden) and FERMOL® (available from DSM Specialties).

In some embodiments, the fermentation composition further comprises an additive. In some embodiments, the additive comprises a surfactant, an antifoaming agent, an antimicrobial agent, a pH adjusting agent (e.g., an acid or a base), a solid support (such as an organic or inorganic solid support), or a processed cell product. In some embodiments, the additive comprises a surfactant. The addition of surfactants can enhance the rate of saccharification. Exemplary surfactants include non-ionic surfactants, such as a Tween® 20 or Tween® 80, polyethylene glycol surfactants, ionic surfactants, detergents, organic solvents, or amphoteric surfactants. In some embodiments, the additive comprises an antifoaming agent, e.g., an oil, an alcohol, a powder, a polyacrylate, a silicon-based agent, or polyglycol (e.g., polyethylene glycol or polypropylene glycol) or polyether (e.g., antifoam 204) dispersions. In some embodiments, the additive comprises an antimicrobial agent, e.g., an antifungal agent (e.g., amphotericin B, fluconazole, micanazole, natamycin, nystatin) or an antibacterial agent (e.g., ampicillin, chloramphenicol, ciprofloxacin, gentamicin, kanamycin, neomycin, penicillin, puromycin, streptomycin). In some embodiments, the additive is a pH adjusting agent, e.g., an acid (e.g., HCl, AcOH, H₂SO₄, H₃PO₄, citric acid, malic acid, succinic acid, or lactic acid) or a base (e.g., NaOH, KOH, Ca(OH)₂, NaHCO₃, CaCO₃, or NH₃). In some embodiments, the additive comprises a processed cell product, e.g., yeast extract, chitin powder, or materials and/or residue from cell culture (e.g., sugar water).

In some embodiments, the fermentation composition further includes supplemental nutrients and chemicals used in addition to the lysed cell matter. These nutrients and chemicals may be added during saccharification and/or fermentation and include, e.g., the food-based nutrient packages described in U.S. Pat. No. 8,852,901, the complete disclosure of which is incorporated herein by reference.

### Further Processing

The present invention described herein provides methods and compositions wherein lysed cell matter is used as an ingredient in the fermentation of biomass (e.g., pretreated biomass, saccharified biomass) to produce a product. In some embodiments, the fermentation products are further processed. For example, the fermentation products (e.g., carbohydrates, organic alcohols, organic acids) can be hydrogenated or treated with other chemicals to produce other products. In some embodiments, hydrogenation can be accomplished by use of a catalyst (e.g., Pt/gamma-Al₂O₃, Ru/C, Raney Nickel, or other catalysts know in the art) in combination with H₂ under high pressure (e.g., 10 to 12000 psi).

In some embodiments, isolation of the fermentation products may involve a distillation step using, for example, a “beer column” to separate ethanol and other alcohols from the majority of water and residual solids. In this case, the vapor exiting the beer column can be, e.g., 35% by weight ethanol and can be fed to a rectification column. A mixture of nearly azeotropic (92.5%) ethanol and water from the rectification column can be purified to pure (99.5%) ethanol using vapor-phase molecular sieves. In some embodiments, the beer column bottoms can be sent to the first effect of a three-effect evaporator. The rectification column reflux condenser can provide heat for this first effect. After the first effect, solids can be separated using a centrifuge and dried in a rotary dryer. A portion (25%) of the centrifuge effluent can be recycled to fermentation and the rest sent to the second and third evaporator effects. Most of the evaporator condensate can be returned to the process as fairly clean condensate with a small portion split off to waste water treatment to prevent build-up of low-boiling compounds.

### Saccharification

The present invention provides methods of producing a product involving the fermentation of biomass (e.g., pretreated biomass, saccharified biomass). In some embodiments, the step immediately prior to fermentation is saccharification. This step involves contacting biomass (e.g., pretreated biomass, biomass exhibiting reduced recalcitrance) to a saccharification composition comprising biomass-degrading enzymes and/or one or more living cells to produce a saccharified biomass. In order to convert the biomass to a form that can be readily processed, the lignocellulosic components of the biomass (e.g., the glucan- or xylan-containing cellulose) can be hydrolyzed to low molecular weight carbohydrates through the use of a saccharification composition comprising biomass-degrading enzymes and/or one or more living cells. The low molecular weight carbohydrates can then be used, for example, for downstream processes including fermentation or other bioprocessing steps.

In some embodiments, the saccharification composition comprises a biomass-degrading enzyme. These isolated enzymes can be supplied by organisms that are capable of breaking down biomass (e.g., the cellulose, hemicellulase, and/or the lignin portions of the biomass), or that contain or manufacture various cellulolytic enzymes (cellulases or xylanases), ligninases or various small molecule biomass-degrading metabolites. In some embodiments, the biomass-degrading enzyme is derived from fungal cells. In some embodiments, the fungal cells comprise a species from the genera Coprinus, Myceliophthora, Scytalidium, Penicillium, Aspergillus, Humicola, Fusarium, Thielavia, Acremonium, Chrysosporium, Saccharomyces, Candida, Clavispora, Pichia, Yarrowia, or Trichoderma. In some embodiments, the fungal cells comprise a species in the genus Trichoderma. In some embodiments, the fungal cells comprise the species Trichoderma reesei. In some embodiments, the Trichoderma reesei comprises any individual strain, variant, or mutant thereof, e.g., Trichoderma reesei QM6a, Trichoderma reesei RL-P37, Trichoderma reesei MCG-80, Trichoderma reesei RUTC30, Trichoderma reesei RUT-NG14, Trichoderma reesei PC3-7, or Trichoderma reesei QM9414. In some embodiments, the Trichoderma reesei comprises strain RUTC30.

In some embodiments, the biomass-degrading enzyme is an endoglucanase, an exoglucanase, a cellobiase, a cellobiohydrolase, a xylanase, a ligninase, or a hemicellulase. In some embodiments, the biomass-degrading enzyme is an endoglucanase, an exoglucanase, a cellobiase, a cellobiohydrolase, a xylanase, a ligninase, or a hemicellulase derived from a fungal cell. In some embodiments, the biomass-degrading enzyme is an endoglucanase, an exoglucanase, a cellobiase, a cellobiohydrolase, a xylanase, a ligninase, or a hemicellulase derived from a species from the genera Coprinus, Myceliophthora, Scytalidium, Penicillium, Aspergillus, Humicola, Fusarium, Thielavia, Acremonium, Chrysosporium, Clostridium, Saccharomyces, Candida, Clavispora, Pichia, Yarrowia, or Trichoderma. In some embodiments, the biomass-degrading enzyme is an endoglucanase, an exoglucanase, a cellobiase, a cellobiohydrolase, a xylanase, a ligninase, or a hemicellulase derived from Trichoderma, e.g., Trichoderma reesei, e.g., any individual strain, variant, or mutant thereof, e.g., Trichoderma reesei QM6a, Trichoderma reesei RL-P37, Trichoderma reesei MCG-80, Trichoderma reesei RUTC30, Trichoderma reesei RUT-NG14, Trichoderma reesei PC3-7, or Trichoderma reesei QM9414. In some embodiments, the biomass-degrading enzyme is a cellobiase, a cellobiohydrolase, a ligninase, or a hemicellulase derived from Trichoderma reesei or any individual strain, variant, or mutant thereof.

In some embodiments, a cellulosic substrate can be initially hydrolyzed during saccharification by endoglucanases at random locations producing oligomeric intermediates. These intermediates are then substrates for exo-splitting glucanases such as cellobiohydrolase to produce cellobiose from the ends of the cellulose polymer. Cellobiose is a water-soluble 1,4-linked dimer of glucose, which may be cleaved into glucose monomers by a cellobiase. The efficiency (e.g., time to hydrolyze and/or completeness of hydrolysis) of this process depends on the recalcitrance of the cellulosic material.

Normally, cellulases and xylanases are used independent of one another. If cellulases are used the product mixture are 6 carbon sugars which, in turn, can be fermented to useful products such as biofuels (e.g., ethanol, butanols). The 6-carbon sugars may be isolated from the hemicellulose. Then independently, the hemicellulose may be converted to useful biochemicals such as L, D lactic acid, succinic acid, furfural products. Or conversely, the xylanase step can be performed first, followed by isolation of the preferred products, and then 6-carbon sugar conversion can be done.

In some embodiments, the saccharification process is carried out in a fluid medium, e.g., an aqueous solution. In some cases, the pretreated biomass is boiled, steeped, or cooked in hot water prior to saccharification, as described in U.S. Patent Publication No. 2012-0100577, the entire contents of which are incorporated herein. In some embodiments, the saccharification process can be partially or completely performed in a tank (e.g., a tank having a volume of at least 4000, 40,000, or 500,000 L) in a manufacturing plant, and/or can be partially or completely performed in transit, e.g., in a rail car, tanker truck, or in a supertanker or the hold of a ship. In some embodiments, the tank is a carbon steel, stainless steel, or ceramic-lined tank. In many embodiments, the tank is configured to control the temperature of the contents within through an apparatus e.g., a jacket, e.g., a steam trace, a half-pipe, or a dimpled jacket. It is generally preferred that the tank contents be mixed during saccharification, e.g., using jet mixing as described in International Application No. PCT/US2010/035331, the full disclosure of which is incorporated by reference herein.

The addition of surfactants can enhance the rate of saccharification. Examples of surfactants include non-ionic surfactants, such as a Tween® 20 or Tween® 80 polyethylene glycol surfactants, ionic surfactants, amphoteric surfactants, detergents, or organic solvents.

In some embodiments, it is generally preferred that the concentration of the sugar solution resulting from saccharification be relatively high, e.g., greater than 40%, or greater than 50, 60, 70, 80, 90 or even greater than 95% by weight. Water may be removed, e.g., by evaporation, to increase the concentration of the sugar solution. This reduces the volume to be shipped, and also inhibits microbial growth in the solution.

Alternatively, sugar solutions of lower concentrations may be used, in which case it may be desirable to add an antimicrobial additive, e.g., a broad spectrum antibiotic, in a low concentration, e.g., 50 to 150 ppm. Other suitable antibiotics include amphotericin B, ampicillin, chloramphenicol, ciprofloxacin, gentamicin, hygromycin B, kanamycin, neomycin, penicillin, puromycin, streptomycin. Antibiotics will inhibit growth of microorganisms during transport and storage, and can be used at appropriate concentrations, e.g., between 15 and 1000 ppm by weight, e.g., between 25 and 500 ppm, or between 50 and 150 ppm. If desired, an antibiotic can be included even if the sugar concentration is relatively high. Alternatively, other additives with anti-microbial of preservative properties may be used. Preferably the antimicrobial additive(s) are food-grade.

A relatively high concentration solution can be obtained by limiting the amount of water added to the biomass material with the enzyme. The concentration can be controlled, e.g., by controlling how much saccharification takes place. For example, concentration can be increased by adding more biomass material to the solution. In order to keep the sugar that is being produced in solution, a surfactant can be added, e.g., as described above. Solubility can also be increased by increasing the temperature of the solution. In some embodiments, the solution can be maintained at a temperature of about 40° C. to about 50° C., about 60° C. to about 80° C., or even higher.

In some embodiments, complete conversion of biomass to a final product is carried out. This process is Simultaneous Saccharification and Fermentation (SSF). Here all of the necessary microorganisms and/or enzymes are added to the biomass (e.g., the pretreated biomass), including the fermentation composition comprising the lysed cell matter, and the conversion occurs in a single reactor or a reactor system. In some embodiments, this process may comprise one conversion that dominates as the slow step, also known as the overall rate determining step. In some embodiments, identification of a target enzyme or target enzymes which will enhance the rate of the slow step (e.g., the rate limiting step) can significantly increase the overall rate of the process. The time required for complete saccharification will depend on the process conditions and the biomass material and enzyme used. For example, if saccharification is performed in a manufacturing plant under controlled conditions, the biomass (e.g., cellulosic or lignocellulosic material) may be substantially entirely converted to sugar (e.g., glucose) in about 12 hours to about 96 hours. However, if saccharification is performed partially or completely in transit, saccharification may take longer.

### Biomass

Provided herein are methods of processing a biomass (e.g., a pretreated biomass, a saccharified biomass) to produce a product. As disclosed herein, biomass (e.g., pretreated biomass, saccharified biomass) is contacted with a fermentation composition comprising lysed cell matter. Biomass materials utilized in the present invention may include lignocellulosic biomass, cellulosic biomass, hemicellulosic biomass, or a combination thereof. Lignocellulosic biomass includes, but is not limited to, wood (e.g., softwood, pine softwood, softwood barks, softwood stems, spruce softwood, hardwood, willow hardwood, aspen hardwood, birch hardwood, hardwood barks, hardwood stems, pine cones, pine needles), particle board, chemical pulps, mechanical pulps, paper, waste paper, forestry wastes (e.g., sawdust, aspen wood, wood chips, leaves), grasses (e.g., switchgrass, miscanthus, cord grass, reed canary grass, coastal Bermuda grass), grain residues (e.g., rice hulls, oat hulls, wheat chaff, barley hulls), agricultural waste (e.g., silage, canola straw, wheat straw, barley straw, oat straw, rice straw, rice bran, jute, hemp, flax, bamboo, sisal, abaca, corn cobs, corn stover, soybean stover, corn fiber, alfalfa, hay, coconut hair, nut shells, palm fronds and palm/coconut oil byproducts), cotton, cotton seed hairs, flax, sugar processing residues (e.g., bagasse, beet pulp, agave bagasse), algae, seaweed, manure (e.g., solid cattle manure, swine waste), sewage, and mixtures of any of these.

Lignocellulosic materials comprise different combinations of cellulose, hemicellulose and lignin. Cellulose is a linear polymer of glucose forming a fairly stiff linear structure without significant coiling. Due to this rigid structure and the disposition of hydroxyl groups available for hydrogen bonding, cellulose contains both crystalline and non-crystalline portions. In some embodiments, the crystalline portions exist as different types, noted as I (alpha) and I (beta), depending on the location of hydrogen bonds between strands. The polymer lengths themselves can vary lending more variety to the form of the cellulose. Hemicellulose is any of several heteropolymers, such as xylan, glucuronoxylan, arabinoxylans, and xyloglucan. The primary sugar monomer present in hemicellulose is xylose, although other monomers such as mannose, galactose, rhamnose, arabinose and glucose are present as well. Typically, hemicellulose forms branched structures with lower molecular weights than cellulose. Hemicellulose is therefore an amorphous material that is generally susceptible to enzymatic hydrolysis. Lignin is a complex high molecular weight heteropolymer. Although all lignins show variability in composition, they have been described as an amorphous dendritic network polymer of phenyl propene units. The amount of cellulose, hemicellulose and lignin in a specific biomaterial depends on the source of the biomaterial. For example, wood derived biomaterial can be about 38% to about 49% cellulose, about 7% to about 26% hemicellulose, and about 23% to about 34% lignin, depending on the type. In contrast, grasses typically comprise about 33% to about 38% cellulose, about 24% to about 32% hemicelluloses, and about 17% to about 22% lignin. Clearly, lignocellulosic biomass constitutes a large class of substrates.

In some embodiments, the lignocellulosic material comprises corncobs. Ground or hammermilled corncobs can be spread in a layer of relatively uniform thickness for pretreatment (e.g., irradiation), and after pretreatment are easy to disperse in the medium for further processing. To facilitate harvest and collection, in some cases the entire corn plant is used, including the corn stalk, corn kernels, and in some cases even the root system of the plant. Corncobs are relatively easy to convey and disperse, and have a lesser tendency to form explosive mixtures in air compared with other cellulosic, hemicellulosic, or lignocellulosic materials upon pretreatment, such as hay and grasses.

In some embodiments, cellulosic biomass includes, for example, paper, paper products, paper waste, paper pulp, pigmented papers, loaded papers, coated papers, filled papers, magazines, printed matter (e.g., books, catalogs, manuals, labels, calendars, greeting cards, brochures, prospectuses, newsprint), printer paper, polycoated paper, card stock, cardboard, paperboard, materials having a high alpha-cellulose content such as cotton, and mixtures of any of these. In some embodiments, cellulosic biomass includes paper products as described in U.S. patent application Ser. No. 13/396,365, the full disclosure of which is incorporated herein by reference. In some embodiments, cellulosic materials can also include lignocellulosic materials which have been partially or fully de-lignified.

In some instances other biomass materials can be utilized, for example, starchy materials. Starchy materials include starch itself, e.g., corn starch, wheat starch, potato starch or rice starch, a derivative of starch, or a material that includes starch, such as an edible food product or a crop. In some embodiments, the starchy material can be arracacha, buckwheat, banana, barley, cassava, kudzu, oca, sago, sorghum, regular household potatoes, sweet potato, taro, yams, or one or more beans, such as favas, lentils or peas. Blends of any two or more starchy materials are also starchy materials. In some embodiments, mixtures of starchy, cellulosic and or lignocellulosic materials can also be used. In some embodiments, a biomass can be an entire plant, a part of a plant or different parts of a plant, e.g., a wheat plant, cotton plant, a corn plant, rice plant or a tree. The starchy materials can be treated by any of the methods described herein.

In some embodiments, biomass may include microbial materials such as any naturally occurring or genetically modified microorganism or organism that contains or is capable of providing a source of carbohydrates (e.g., cellulose), for example, protists (e.g., animal protists (e.g., protozoa such as flagellates, amoeboids, ciliates, and sporozoa) and plant protists (e.g., algae such alveolates, chlorarachniophytes, cryptomonads, euglenids, glaucophytes, haptophytes, red algae, stramenopiles, and viridiplantae)). Other examples include seaweed, plankton (e.g., macroplankton, mesoplankton, microplankton, nanoplankton, picoplankton, and femtoplankton), phytoplankton, bacteria (e.g., gram positive bacteria, gram negative bacteria, and extremophiles), yeast and/or mixtures of these. In some instances, microbial biomass can be obtained from natural sources, e.g., the ocean, lakes, bodies of water, e.g., salt water or fresh water, or on land. Alternatively or in addition, microbial biomass can be obtained from culture systems, e.g., large scale dry and wet culture and fermentation systems.

In other embodiments, the biomass materials, such as cellulosic, hemicellulosic, starchy and lignocellulosic feedstock materials, can be obtained from transgenic microorganisms and plants that have been modified with respect to a wild type variety. Such modifications may be, for example, through the iterative steps of selection and breeding to obtain desired traits in a plant. Furthermore, the plants can have had genetic material removed, modified, silenced and/or added with respect to the wild type variety. For example, genetically modified plants can be produced by recombinant DNA methods, where genetic modifications include introducing or modifying specific genes from parental varieties, or, for example, by using transgenic breeding wherein a specific gene or genes are introduced to a plant from a different species of plant and/or bacteria. Another way to create genetic variation is through mutation breeding wherein new alleles are artificially created from endogenous genes. The artificial genes can be created by a variety of ways including treating the plant or seeds with, for example, chemical mutagens (e.g., using alkylating agents, epoxides, alkaloids, peroxides, formaldehyde), irradiation (e.g., X-rays, gamma rays, neutrons, beta particles, alpha particles, protons, deuterons, UV radiation) and temperature shocking or other external stressing and subsequent selection techniques. Other methods of providing modified genes is through error prone PCR and DNA shuffling followed by insertion of the desired modified DNA into the desired plant or seed. Methods of introducing the desired genetic variation in the seed or plant include, for example, the use of a bacterial carrier, biolistics, calcium phosphate precipitation, electroporation, gene splicing, gene silencing, lipofection, microinjection and viral carriers. Additional genetically modified materials have been described in U.S. patent application Ser. No. 13/396,369, the full disclosure of which is incorporated herein by reference.

In some embodiments, the biomass material can also include offal, and similar sources of material. In some embodiments, any of the methods described herein can be practiced with mixtures of any biomass materials described herein.

### Treatment of Biomass

In general, the invention relates to improvements in processing biomass materials (e.g., biomass materials or biomass-derived materials) to produce intermediates and products, such as food, biochemicals, biofuels, or other products. Biomass is considered any mixture comprising cellulose, hemicellulose and lignin. In some embodiments, the invention described herein may be used to produce sugars, alcohols (e.g., ethanol, isobutanol, or n-butanol), sugar alcohols (such as xylitol, erythritol), or organic acids (e.g., lactic acid, succinic acid, lactic acid.)

In some embodiments, the process of producing food, biochemicals, and biofuels from biomass involves consideration of several distinct components. In some embodiments, these components include at least: a) the source of the biomass, b) the composition of the biomass, c) the method of pretreating of the biomass, d) saccharification, e) fermentation, and optionally f) isolation of products. Each of these various steps can be optimized to achieve highest possible yields of the desired products. In some embodiments, optimization of one step may require addition of another step in the overall process to prevent a negative impact on a downstream process. For instance, if during the pretreatment step, it is deemed to be advantageous to use an acid to facilitate biomass degradation, then a neutralization step may be added to the overall process in order to prevent negatively affecting the fermentation step.

In embodiments, reducing the recalcitrance of the biomass includes treating the cellulose, hemicellulose and/or lignocellulose materials with a physical treatment. The physical treatment can be, for example, radiation (e.g., electron bombardment), sonication, pyrolysis, oxidation, steam explosion, chemical treatment, heat treatment, or combinations of any of these treatments. The treatments can also include any one or more of the treatments disclosed herein, applied alone or in any desired combination, and applied once or multiple times. In some embodiments, these steps can include an additional pretreatment step which reduces the size of the pieces of the biomass to a size that can be easily conveyed to the treatment step. The pretreatment step is thought to involve physical reduction in size and narrow the size distribution of the biomass particles. The treatment step can include disrupting some of the chemical bonding in biomass leading to material that has reduced recalcitrance. This treatment step can perform a physical/chemical step that might begin to separate the various components of the biomass from each other. This, in turn, can lead to improved saccharification in the subsequent step.

The diversity of biomass materials may be further increased by pretreatment, for example, by changing the physical size, the crystallinity, and molecular weight of the polymers. In some embodiments, the pretreatment and treatment conditions can lead to molecular changes. For example, as the lignin is separated or cleaved from the cellulose and/or hemicellulose fragments of phenyl propene can be released that can lead to inhibition in subsequent steps involving microorganisms.

### Mechanical Treatment of Biomass

In some embodiments, the biomass can undergo several processing steps prior to the fermentation step in which the lysed cell matter is added. In some embodiments, the first step involves reduction of the overall size of the biomass. This commutation step is described below and can be called a pretreatment step relative to the recalcitrance reduction step described next. The next step, the treatment step, is usually the most effective step in reducing the recalcitrance of the biomass materials and can be any of: irradiation, especially bombardment with electrons, sonication, oxidation, pyrolysis, steam explosion, ammonia treatments, chemical treatment, heat treatment, mechanical treatment, and freeze grinding and combinations thereof. Preferably, the treatment method is bombardment with electrons. This irradiation with electrons is often coupled to the other pretreatments and treatments described herein and can include use of a conveyor to move the biomass between operations.

In some embodiments, the biomass can be in a dry form, for example, with less than about 35% moisture content (e.g., less than about 20%, less than about 15%, less than about 10% less than about 5%, less than about 4%, less than about 3%, less than about 2% or even less than about 1%). In some embodiments, the biomass can be delivered in a wet state, for example, as a wet solid, a slurry or a suspension with at least about 10 wt. % solids (e.g., at least about 20 wt. %, at least about 30 wt. %, at least about 40 wt. %, at least about 50 wt. %, at least about 60 wt. %, at least about 70 wt. %). This moisture content is determined measured at 25° C. and at fifty percent relative humidity.

In some embodiments, the processes disclosed herein can utilize low bulk density materials, for example, cellulosic or lignocellulosic biomass that has been physically pretreated to have a bulk density of less than about 0.75 g/cm³, e.g., less than about 0.7 g/cm³, 0.65 g/cm³, 0.60 g/cm³, 0.50 g/cm³, 0.35 g/cm³, 0.25 g/cm³, 0.20 g/cm³, 0.15 g/cm³, 0.10 g/cm³, 0.05 g/cm³ or less, e.g., less than about 0.025 g/cm³. In some embodiments, bulk density is determined using ASTM D1895B. Briefly, the method involves filling a measuring cylinder of known volume with a sample and obtaining a weight of the sample. The bulk density is calculated by dividing the weight of the sample in grams by the known volume of the cylinder in cubic centimeters. If desired, low bulk density materials can be densified, for example, by methods described in U.S. Pat. No. 7,971,809, the full disclosure of which is hereby incorporated by reference.

In some embodiments, the pretreatment processing includes screening of the biomass material and using a conveyor to move the biomass material from one pretreatment to another processing step. In some embodiments, screening can be through a mesh or perforated plate with a desired opening size, for example, less than about 6.35 mm (¼ inch, 0.25 inch), (e.g., less than about 3.18 mm (⅛ inch, 0.125 inch), less than about 1.59 mm ( 1/16 inch, 0.0625 inch), is less than about 0.79 mm ( 1/32 inch, 0.03125 inch), e.g., less than about 0.51 mm ( 1/50 inch, 0.02000 inch), less than about 0.40 mm ( 1/64 inch, 0.015625 inch), less than about 0.23 mm (0.009 inch), less than about 0.20 mm ( 1/128 inch, 0.0078125 inch), less than about 0.18 mm (0.007 inch), less than about 0.13 mm (0.005 inch), or even less than about 0.10 mm ( 1/256 inch, 0.00390625 inch)).

Screening of material can also be by a manual method, for example by an operator or mechanoid (e.g., a robot equipped with a color, reflectivity or other sensor) that removes unwanted material. Screening can also be by magnetic screening wherein a magnet is disposed near the conveyed material and the magnetic material is removed magnetically.

The material can be leveled to form a uniform thickness between about 0.0312 and 5 inches (e.g., between about 0.0625 and 2.000 inches, between about 0.125 and 1 inches, between about 0.125 and 0.5 inches, between about 0.3 and 0.9 inches, between about 0.2 and 0.5 inches between about 0.25 and 1.0 inches, between about 0.25 and 0.5 inches, 0.100+/−0.025 inches, 0.150+/−0.025 inches, 0.200+/−0.025 inches, 0.250+/−0.025 inches, 0.300+/−0.025 inches, 0.350+/−0.025 inches, 0.400+/−0.025 inches, 0.450+/−0.025 inches, 0.500+/−0.025 inches, 0.550+/−0.025 inches, 0.600+/−0.025 inches, 0.700+/−0.025 inches, 0.750+/−0.025 inches, 0.800 +/−0.025 inches, 0.850+/−0.025 inches, 0.900+/−0.025 inches, 0.900+/−0.025 inches.

In some cases, the mechanical treatment may include an initial preparation of the biomass as received, e.g., size reduction of materials, such as by comminution, e.g., cutting, grinding, shearing, pulverizing or chopping. For example, in some cases, loose feedstock (e.g., recycled paper, starchy materials, or switchgrass) is prepared by shearing or shredding. Mechanical treatment may reduce the bulk density of the carbohydrate-containing material, increase the surface area of the carbohydrate-containing material and/or decrease one or more dimensions of the carbohydrate-containing material.

In addition to size reduction, which can be performed initially and/or later in processing, mechanical treatment can also be advantageous for “opening up,” “stressing,” breaking or shattering the carbohydrate-containing materials, making the cellulose of the materials more susceptible to chain scission and/or disruption of crystalline structure during the physical treatment.

In some embodiments, methods of mechanically treating the carbohydrate-containing material include, for example, milling or grinding. Milling may be performed using, for example, a hammer mill, ball mill, colloid mill, conical or cone mill, disk mill, edge mill, Wiley mill, grist mill or other mill. Grinding may be performed using, for example, a cutting/impact type grinder. Some exemplary grinders include stone grinders, pin grinders, coffee grinders, and burr grinders. Grinding or milling may be provided, for example, by a reciprocating pin or other element, as is the case in a pin mill. Other mechanical treatment methods include mechanical ripping or tearing, other methods that apply pressure to the fibers, and air attrition milling. Suitable mechanical treatments further include any other technique that continues the disruption of the internal structure of the material that was initiated by the previous processing steps.

The milling of the biomass may be done either in a wet or dry state. The optimum condition can depend on the milling equipment, the biomass, whether subsequent steps are more suited to processing a dry material. The preferred liquid for the wet milling is water, and this can be done without additives like sulfur dioxide. Dry milling of the biomass may be a preferred process especially if subsequent treatments are better done is a dry state where the water content is less than about 15 weight percent, optionally less than 10 weight percent, or alternatively less than 5 weight percent. For example, the material can be wet and/or dry milled by the methods and equipment disclosed in U.S. Pat. No. 7,900,857, U.S. Pat. No. 8,420,356, and U.S. Patent Publication 2012/0315675, the full disclosures of which are incorporated herein by reference.

In some embodiments, mechanical feed preparation systems can be configured to produce streams with specific characteristics such as, for example, specific maximum sizes, specific length-to-width, or specific surface areas ratios. Physical preparation can increase the rate of reactions, improve the movement of material on a conveyor, improve the irradiation profile of the material, improve the radiation uniformity of the material, or reduce the processing time required by opening up the materials and making them more accessible to processes and/or reagents, such as reagents in a solution.

In some embodiments, the bulk density of feedstocks can be controlled (e.g., increased). In some situations, it can be desirable to prepare a low bulk density material, e.g., by densifying the material (e.g., densification can make it easier and less costly to transport to another site) and then reverting the material to a lower bulk density state (e.g., after transport). The material can be densified, for example, from less than about 0.2 g/cc to more than about 0.9 g/cc (e.g., less than about 0.3 to more than about 0.5 g/cc, less than about 0.3 to more than about 0.9 g/cc, less than about 0.5 to more than about 0.9 g/cc, less than about 0.3 to more than about 0.8 g/cc, less than about 0.2 to more than about 0.5 g/cc). For example, the material can be densified by the methods and equipment disclosed in U.S. Pat. No. 7,932,065 and International Publication No. WO 2008/073186, the full disclosures of which are incorporated herein by reference. Densified materials can be processed by any of the methods described herein, or any material processed by any of the methods described herein can be subsequently densified.

In some embodiments, the material to be processed is in the form of a fibrous material that includes fibers provided by shearing a fiber source. In some embodiments, the shearing can be performed with a rotary knife cutter. In some embodiments, a fiber source, e.g., that is recalcitrant or that has had its recalcitrance level reduced, can be sheared, e.g., in a rotary knife cutter, to provide a first fibrous material. The first fibrous material is passed through a first screen, e.g., having an average opening size of 1.59 mm or less ( 1/16 inch, 0.0625 inch), provide a second fibrous material. If desired, the fiber source can be cut prior to the shearing, e.g., with a shredder. For example, when a paper is used as the fiber source, the paper can be first cut into strips that are, e.g., ¼- to ½-inch wide, using a shredder, e.g., a counter-rotating screw shredder, such as those manufactured by Munson (Utica, N.Y.). As an alternative to shredding, the paper can be reduced in size by cutting to a desired size using a guillotine cutter. For example, the guillotine cutter can be used to cut the paper into sheets that are, e.g., 10 inches wide by 12 inches long.

In some embodiments, the shearing of the fiber source and the passing of the resulting first fibrous material through a first screen are performed concurrently. The shearing and the passing can also be performed in a batch-type process. For example, a rotary knife cutter can be used to concurrently shear the fiber source and screen the first fibrous material. A rotary knife cutter includes a hopper that can be loaded with a shredded fiber source prepared by shredding a fiber source.

Mechanical treatments that may be used, and the characteristics of the mechanically treated carbohydrate-containing materials, are described in further detail in U.S. Patent Publication No. 2012/0100577, the full disclosure of which is hereby incorporated herein by reference.

### Heat Treatment of Biomass

In some embodiments, the biomass may be heat treated for up to twelve hours at temperatures ranging from about 90° C. to about 160° C. In some embodiments, this heat treatment step is performed in conjunction with or after another treatment step (e.g., irradiation). In some embodiments, the amount of time for the heat treatment is up to 9 hours, alternately up to 6 hours, optionally up to 4 hours and further up to about 2 hours. The treatment time can be up to as little as 30 minutes when the mass may be effectively heated.

In some embodiments, the heat treatment can be performed 90° C. to about 160° C. or, optionally, at 100° C. to 150° C. or, alternatively, at 120° C. to 140° C. In some embodiments, the heat treatment is performed in an aqueous suspension or mixture of the biomass. The amount of biomass is 10 to 90 wt. % of the total mixture, alternatively 20 to 70 wt. % or optionally 25 to 50 wt. %. The irradiated biomass may have minimal water content so water must be added prior to the heat treatment.

Since at temperatures above 100° C. there will be pressure due at least in part to the vaporization of water, a pressure vessel can be utilized to accommodate and/or maintain the pressure. In some embodiments, the process for the heat treatment may be batch, continuous, semi-continuous or other reactor configurations. The continuous reactor configuration may be a tubular reactor and may include device(s) within the tube which will facilitate heat transfer and mixing/suspension of the biomass. These tubular devices may include a one or more static mixers. The heat may also be put into the system by direct injection of steam.

In some embodiments, a portion of a conveyor conveying the biomass or other material can be sent through a heated zone. The heated zone can be created, for example, by IR radiation, microwaves, combustion (e.g., gas, coal, oil, biomass), resistive heating and/or inductive coils. The heat can be applied from at least one side or more than one side, can be continuous or periodic and can be for only a portion of the material or all the material. For example, a portion of the conveying trough can be heated by use of a heating jacket. Heating can be, for example, for the purpose of drying the material. In the case of drying the material, this can also be facilitated, with or without heating, by the movement of a gas (e.g., air, oxygen, nitrogen, He, CO2, Argon) over and/or through the biomass as it is being conveyed.

In some embodiments, pretreatment processing of the biomass can include cooling the material. Cooling material is described in U.S. Pat. No. 7,900,857, the disclosure of which in incorporated herein by reference. In some embodiments, cooling can be by supplying a cooling fluid, for example, water (e.g., with glycerol), or nitrogen (e.g., liquid nitrogen) to the bottom of the conveying trough. In some embodiments, a cooling gas, for example, chilled nitrogen can be blown over the biomass materials or under the conveying system.

### Radiation Treatment of Biomass

In some embodiments, reducing the recalcitrance of the biomass includes treating the cellulose, hemicellulose and/or lignocellulose materials with a physical treatment. In some embodiments, the biomass (e.g., cellulosic, hemicellulose, and lignocellulosic biomass) can be treated with electron bombardment to modify its structure, for example, to reduce its recalcitrance or cross link the structures. Such treatment can, for example, reduce the average molecular weight of the feedstock, change the crystalline structure of the feedstock, and/or increase the surface area and/or porosity of the feedstock.

In some embodiments, a beam of electrons can be used as the radiation source. A beam of electrons has the advantages of high dose rates (e.g., 1, 5, or even 10 Mrad per second), high throughput, less containment, and less confinement equipment. Electron beams can also have high electrical efficiency (e.g., 80%), allowing for lower energy usage relative to other radiation methods, which can translate into a lower cost of operation and lower greenhouse gas emissions corresponding to the smaller amount of energy used. Electron beams can be generated, e.g., by electrostatic generators, cascade generators, transformer generators, low energy accelerators with a scanning system, low energy accelerators with a linear cathode, linear accelerators, and pulsed accelerators.

Electrons can also be more efficient at causing changes in the molecular structure of carbohydrate-containing materials, for example, by the mechanism of chain scission. In addition, electrons having energies of 0.5-10 MeV can penetrate low density materials, such as the biomass materials described herein, e.g., materials having a bulk density of less than 0.5 g/cm3, and a depth of 0.3-10 cm. Electrons as an ionizing radiation source can be useful, e.g., for relatively thin piles, layers or beds of materials, e.g., less than about 0.5 inch, e.g., less than about 0.4 inch, 0.3 inch, 0.25 inch, or less than about 0.1 inch. In some embodiments, the energy of each electron of the electron beam is from about 0.3 MeV to about 2.0 MeV (million electron volts), e.g., from about 0.5 MeV to about 1.5 MeV, or from about 0.7 MeV to about 1.25 MeV. Methods of irradiating materials are discussed in U.S. Patent Publication 2012/0100577 A1, the entire disclosure of which is herein incorporated by reference.

In some embodiments, radiation can be provided by, for example, electron beam, ion beam, 100 nm to 28 nm ultraviolet (UV) light, gamma or X-ray radiation. Radiation treatments and systems for treatments are discussed in U.S. Pat. No. 8,142,620, and U.S. patent application Ser. No. 12/417,731, the entire disclosures of which are incorporated herein by reference. In some embodiments, radiation treatment of biomass can produce radicals that can be sites for cross-linking, grafting and/or functionalization.

Each form of radiation ionizes the biomass via particular interactions, as determined by the energy of the radiation. Heavy charged particles primarily ionize matter via Coulomb scattering; furthermore, these interactions produce energetic electrons that may further ionize matter. Alpha particles are identical to the nucleus of a helium atom and are produced by the alpha decay of various radioactive nuclei, such as isotopes of bismuth, polonium, astatine, radon, francium, radium, several actinides, such as actinium, thorium, uranium, neptunium, curium, californium, americium, and plutonium. Electrons interact via Coulomb scattering and bremsstrahlung radiation produced by changes in the velocity of electrons.

When particles are utilized, they can be neutral (uncharged), positively charged or negatively charged. When charged, the charged particles can bear a single positive or negative charge, or multiple charges, e.g., one, two, three or even four or more charges. In instances in which chain scission is desired to change the molecular structure of the carbohydrate containing material, positively charged particles may be desirable, in part, due to their acidic nature. When particles are utilized, the particles can have the mass of a resting electron, or greater, e.g., 500, 1000, 1500, or 2000 or more times the mass of a resting electron. For example, the particles can have a mass of from about 1 atomic unit to about 150 atomic units, e.g., from about 1 atomic unit to about 50 atomic units, or from about 1 to about 25, e.g., 1, 2, 3, 4, 5, 10, 12 or 15 atomic units.

Gamma radiation has the advantage of a significant penetration depth into a variety of material in the sample.

In embodiments in which the irradiating is performed with electromagnetic radiation, the electromagnetic radiation can have, e.g., energy per photon (in electron volts) of greater than 10² eV, e.g., greater than 10³, 10⁴, 10⁵, 10⁶, or even greater than 10⁷ eV. In some embodiments, the electromagnetic radiation has energy per photon of between 10⁴ and 10⁷, e.g., between 10⁵ and 10⁶ eV. The electromagnetic radiation can have a frequency of, e.g., greater than 10¹⁶ Hz, greater than 10¹⁷ Hz, 10¹⁸, 10¹⁹, 10²⁰, or even greater than 10²¹ Hz. In some embodiments, the electromagnetic radiation has a frequency of between 10¹⁸ and 10 ²²Hz, e.g., between 10¹⁹ to 10²¹ Hz.

In some embodiments, radiation treatment is performed with electron bombardment. In some embodiments, electron bombardment may be performed using an electron beam device that has a nominal energy of less than 10 MeV, e.g., less than 7 MeV, less than 5 MeV, or less than 2 MeV, e.g., from about 0.5 to 1.5 MeV, from about 0.8 to 1.8 MeV, or from about 0.7 to 1 MeV. In some implementations the nominal energy is about 500 to 800 keV. The electron beam may have a relatively high total beam power (the combined beam power of all accelerating heads, or, if multiple accelerators are used, of all accelerators and all heads), e.g., at least 25 kW, e.g., at least 30, 40, 50, 60, 65, 70, 80, 100, 125, or 150, 250, 300 kW. In some cases, the power is even as high as 500 kW, 750 kW, or even 1000 kW or more. In some cases the electron beam has a beam power of 1200 kW or more, e.g., 1400, 1600, 1800, or even 3000 kW. The electron beam may have a total beam power of 25 to 3000 kW. Alternatively, the electron beam may have a total beam power of 75 to 1500 kW. Optionally, the electron beam may have a total beam power of 100 to 1000 kW. Alternatively, the electron beam may have a total beam power of 100 to 400 kW.

In some embodiments, it is desirable to treat the material with radiation as quickly as possible. In general, it is preferred that treatment be performed at a dose rate of greater than about 0.25 Mrad per second, e.g., greater than about 0.5, 0.75, 1, 1.5, 2, 5, 7, 10, 12, 15, or even greater than about 20 Mrad per second, e.g., about 0.25 to 30 Mrad per second. In some embodiments, the treatment is performed at a dose rate of 0.5 to 20 Mrad per second. In some embodiments, the treatment is performed at a dose rate of 0.75 to 15 Mrad per second. In some embodiments, the treatment is performed at a dose rate of 1 to 5 Mrad per second. In some embodiments, the treatment is performed at a dose rate of 1-3 Mrad per second or alternatively 1-2 Mrad per second. Higher dose rates allow a higher throughput for a target (e.g., the desired) dose. Higher dose rates generally require higher line speeds, to avoid thermal decomposition of the material. In one implementation, the accelerator is set for 3 MeV, 50 mA beam current, and the line speed is 24 feet/minute, for a sample thickness of about 20 mm (e.g., comminuted corn cob material with a bulk density of 0.5 g/cm³).

In some embodiments, electron bombardment is performed until the material receives a total dose of at least 0.1 Mrad, 0.25 Mrad, 1 Mrad, 5 Mrad, e.g., at least 10, 20, 30 or at least 40 Mrad. In some embodiments, the treatment is performed until the material receives a dose of from about 10 Mrad to about 50 Mrad, e.g., from about 20 Mrad to about 40 Mrad, or from about 25 Mrad to about 30 Mrad. In some implementations, a total dose of 25 to 35 Mrad is preferred, applied ideally over a couple of passes, e.g., at 5 Mrad/pass with each pass being applied for about one second. Cooling methods, systems and equipment can be used before, during, after and in between radiations, for example, utilizing a cooling screw conveyor and/or a cooled vibratory conveyor.

In some embodiments, using multiple beam heads allows for the material can be treated in multiple passes, for example, two passes at 10 to 20 Mrad/pass, e.g., 12 to 18 Mrad/pass, separated by a few seconds of cool-down, or three passes of 7 to 12 Mrad/pass, e.g., 5 to 20 Mrad/pass, 10 to 40 Mrad/pass, 9 to 11 Mrad/pass. As discussed herein, treating the material with several relatively low doses, rather than one high dose, tends to prevent overheating of the material and also increases dose uniformity through the thickness of the material. In some implementations, the material is stirred or otherwise mixed during or after each pass and then smoothed into a uniform layer again before the next pass, to further enhance treatment uniformity.

In some embodiments, two or more electron sources are used, such as two or more ionizing sources. For example, samples can be treated, in any order, with a beam of electrons, followed by gamma radiation and UV light having wavelengths from about 100 nm to about 280 nm. In some embodiments, samples are treated with three ionizing radiation sources, such as a beam of electrons, gamma radiation, and energetic UV light. The biomass is conveyed through the treatment zone where it can be bombarded with electrons.

The effectiveness in changing the molecular/supermolecular structure and/or reducing the recalcitrance of the carbohydrate-containing biomass depends on the electron energy used and the dose applied, while exposure time depends on the power and dose. In some embodiments, the dose rate and total are adjusted so as not to destroy (e.g., char or burn) the biomass material. For example, the carbohydrates should not be damaged in the processing so that they can be released from the biomass intact, e.g. as monomeric sugars.

It also can be desirable to irradiate from multiple directions, simultaneously or sequentially, in order to achieve a desired degree of penetration of radiation into the material. For example, depending on the density and moisture content of the material, such as wood, and the type of radiation source used (e.g., gamma or electron beam), the maximum penetration of radiation into the material may be only about 0.75 inch. In such a cases, a thicker section (up to 1.5 inch) can be irradiated by first irradiating the material from one side, and then turning the material over and irradiating from the other side. Irradiation from multiple directions can be particularly useful with electron beam radiation, which irradiates faster than gamma radiation but typically does not achieve as great a penetration depth.

The type of radiation determines the kinds of radiation sources used as well as the radiation devices and associated equipment. The methods, systems and equipment described herein, for example, for treating materials with radiation, can utilized sources as described herein as well as any other useful source. In some embodiments, sources of gamma rays include radioactive nuclei, such as isotopes of cobalt, calcium, technetium, chromium, gallium, indium, iodine, iron, krypton, samarium, selenium, sodium, thallium, and xenon. In some embodiments, sources of X-rays include electron beam collision with metal targets, such as tungsten or molybdenum or alloys, or compact light sources, such as those produced commercially by Lyncean. In some embodiments, alpha particles are identical to the nucleus of a helium atom and are produced by the alpha decay of various radioactive nuclei, such as isotopes of bismuth, polonium, astatine, radon, francium, radium, several actinides, such as actinium, thorium, uranium, neptunium, curium, californium, americium, and plutonium. In some embodiments, sources for ultraviolet radiation include deuterium or cadmium lamps. In some embodiments, sources for infrared radiation include sapphire, zinc, or selenide window ceramic lamps. In some embodiments, sources for microwaves include klystrons, Slevin type RF sources, or atom beam sources that employ hydrogen, oxygen, or nitrogen gases.

In some embodiments, accelerators used to accelerate the particles can be electrostatic DC, electrodynamic DC, RF linear, magnetic induction linear or continuous wave. For example, cyclotron type accelerators are available from IBA, Belgium, such as the RHODOTRON™ system, while DC type accelerators are available from RDI, now IBA Industrial, such as the DYNAMITRON®. Ions and ion accelerators are discussed in Introductory Nuclear Physics, Kenneth S. Krane, John Wiley & Sons, Inc. (1988), Krsto Prelec, FIZIKA B 6 (1997) 4, 177-206, Chu, William T., “Overview of Light-Ion Beam Therapy”, Columbus-Ohio, ICRU-IAEA Meeting, 18-20 Mar. 2006, Iwata, Y. et al., “Alternating-Phase-Focused IH-DTL for Heavy-Ion Medical Accelerators”, Proceedings of EPAC 2006, Edinburgh, Scotland, and Leitner, C. M. et al., “Status of the Superconducting ECR Ion Source Venus”, Proceedings of EPAC 2000, Vienna, Austria.

In some embodiments, electrons may be produced by radioactive nuclei that undergo beta decay, such as isotopes of iodine, cesium, technetium, and iridium. Alternatively, an electron gun can be used as an electron source via thermionic emission and accelerated through an accelerating potential. An electron gun generates electrons, which are then accelerated through a large potential (e.g., greater than about 500 thousand, greater than about 1 million, greater than about 2 million, greater than about 5 million, greater than about 6 million, greater than about 7 million, greater than about 8 million, greater than about 9 million, or even greater than 10 million volts) and then scanned magnetically in the x-y plane, where the electrons are initially accelerated in the z direction down the accelerator tube and extracted through a foil window. Scanning the electron beams is useful for increasing the irradiation surface when irradiating materials, e.g., a biomass, that is conveyed through the scanned beam. Scanning the electron beam also distributes the thermal load homogenously on the window and helps reduce the foil window rupture due to local heating by the electron beam. Window foil rupture is a cause of significant down-time due to subsequent necessary repairs and re-starting the electron gun.

In some embodiments, various other irradiating devices may be used in the methods disclosed herein, including field ionization sources, electrostatic ion separators, field ionization generators, thermionic emission sources, microwave discharge ion sources, recirculating or static accelerators, dynamic linear accelerators, van de Graaff accelerators, and folded tandem accelerators. Such devices are disclosed, for example, in U.S. Pat. No. 7,931,784 to Medoff, the complete disclosure of which is incorporated herein by reference.

In some embodiments, electron beam irradiation devices may be procured commercially from Ion Beam Applications, Louvain-la-Neuve, Belgium, NHV Corporation, Japan or the Titan Corporation, San Diego, Calif. Typical electron energies can be 0.5 MeV, 1 MeV, 2 MeV, 4.5 MeV, 7.5 MeV, or 10 MeV. Typical electron beam irradiation device power can be 1 kW, 5 kW, 10 kW, 20 kW, 50 kW, 60 kW, 70 kW, 80 kW, 90 kW, 100 kW, 125 kW, 150 kW, 175 kW, 200 kW, 250 kW, 300 kW, 350 kW, 400 kW, 450 kW, 500 kW, 600 kW, 700 kW, 800 kW, 900 kW or even 1000 kW.

Tradeoffs in considering electron beam irradiation device power specifications include cost to operate, capital costs, depreciation, and device footprint. Tradeoffs in considering exposure dose levels of electron beam irradiation would be energy costs and environment, safety, and health (ESH) concerns. Typically, generators are housed in a vault, e.g., of lead or concrete, especially for production from X-rays that are generated in the process. Tradeoffs in considering electron energies include energy costs. The electron beam irradiation device can produce either a fixed beam or a scanning beam. A scanning beam may be advantageous with large scan sweep length and high scan speeds, as this would effectively replace a large, fixed beam width. Further, available sweep widths of 0.5 m, 1 m, 2 m or more are available. The scanning beam is preferred in most embodiments describe herein because of the larger scan width and reduced possibility of local heating and failure of the windows.

Several processes can occur in biomass when electrons from an electron beam interact with matter in inelastic collisions. For example, ionization of the material, chain scission of polymers in the material, cross linking of polymers in the material, oxidation of the material, generation of X-rays (“Bremsstrahlung”) and vibrational excitation of molecules (e.g. phonon generation). Without being bound to a particular mechanism, the reduction in recalcitrance can be due to several of these inelastic collision effects, for example, ionization, chain scission of polymers, oxidation and phonon generation. Some of the effects (e.g., especially X-ray generation), necessitate shielding and engineering barriers, for example, enclosing the irradiation processes in a concrete (or other radiation opaque material) vault. Another effect of irradiation, vibrational excitation, is equivalent to heating up the sample. Heating the sample by irradiation can help in recalcitrance reduction, but excessive heating can destroy the material, as will be explained below.

The adiabatic temperature rise (ΔT) from adsorption of ionizing radiation is given by the equation: ΔT=D/Cp: where D is the average dose in KGy, Cp is the heat capacity in J/g ° C., and ΔT is the change in temperature in ° C. A typical dry biomass material will have a heat capacity close to 2. Wet biomass will have a higher heat capacity dependent on the amount of water since the heat capacity of water is very high (4.19 J/g ° C.). Metals have much lower heat capacities, for example, 304 stainless steel has a heat capacity of 0.5 J/g ° C. The temperature change due to the instant adsorption of radiation in a biomass and stainless steel for various doses of radiation is shown in Table 1.

TABLE 1
Calculated Temperature increase for
biomass and stainless steel.

| Dose | Estimated | Steel |
|---|---|---|
| (Mrad) | Biomass ΔT (° C.) | ΔT (° C.) |
| 10 | 50 | 200 |
| 50 | 250, Decomposition | 1000 |
| 100 | 500, Decomposition | 2000 |
| 150 | 750, Decomposition | 3000 |
| 200 | 1000, Decomposition | 4000 |

High temperatures can destroy and or modify the biopolymers in biomass so that the polymers (e.g., cellulose) are unsuitable for further processing. A biomass subjected to high temperatures can become dark, sticky and give off odors indicating decomposition. The stickiness can even make the material hard to convey. The odors can be unpleasant and be a safety issue. In fact, keeping the biomass below about 200° C. has been found to be beneficial in the processes described herein (e.g., below about 190° C., below about 180° C., below about 170° C., below about 160° C., below about 150° C., below about 140° C., below about 130° C., below about 120° C., below about 110° C., between about 60° C. and 180° C., between about 60° C. and 160° C., between about 60° C. and 150° C., between about 60° C. and 140° C., between about 60° C. and 130° C., between about 60° C. and 120° C., between about 80° C. and 180° C., between about 100° C. and 180° C., between about 120° C. and 180° C., between about 140° C. and 180° C., between about 160° C. and 180° C., between about 100° C. and 140° C., between about 80° C. and 120° C.).

It has been found that irradiation above about 10 Mrad is desirable for the processes described herein (e.g., reduction of recalcitrance). A high throughput is also desirable so that the irradiation does not become a bottle neck in processing the biomass. The treatment is governed by a Dose rate equation: M=FP/D * time, where M is the mass of irradiated material (Kg), F is the fraction of power that is adsorbed (unit less), P is the emitted power (KW=Voltage in MeV * Current in mA), time is the treatment time (sec) and D is the adsorbed dose (KGy). In an exemplary process where the fraction of adsorbed power is fixed, the Power emitted is constant and a set dosage is desired, the throughput (e.g., M, the biomass processed) can be increased by increasing the irradiation time. However, increasing the irradiation time without allowing the material to cool, can excessively heat the material as exemplified by the calculations shown above. Since biomass has a low thermal conductivity (less than about 0.1 Wm⁻¹K⁻¹), heat dissipation is slow, unlike, for example metals (greater than about 10 Wm⁻¹K⁻¹) which can dissipate energy quickly as long as there is a heat sink to transfer the energy to.

In some embodiments, the systems and methods include a beam stop (e.g., a shutter). For example, the beam stop can be used to quickly stop or reduce the irradiation of material without powering down the electron beam device. Alternatively the beam stop can be used while powering up the electron beam, e.g., the beam stop can stop the electron beam until a beam current of a desired level is achieved. The beam stop can be placed between the primary foil window and a secondary foil window. For example, the beam stop can be mounted so that it is movable, that is, so that it can be moved into and out of the beam path. Even partial coverage of the beam can be used, for example, to control the dose of irradiation. The beam stop can be mounted to the floor, to a conveyor for the biomass, to a wall, to the radiation device (e.g., at the scan horn), or to any structural support. Preferably the beam stop is fixed in relation to the scan horn so that the beam can be effectively controlled by the beam stop. The beam stop can incorporate a hinge, a rail, wheels, slots, or other means allowing for its operation in moving into and out of the beam. The beam stop can be made of any material that will stop at least 5% of the electrons, e.g., at least 10%, 20%, 30%, 40%, 50%, 60%, 70%, at least 80%, 85%, 90%, 91%, 92%, 93%, 94%, 95%, 96%, 97%, 98%, 99% or even about 100% of the electrons.

In some embodiments, the beam stop can be made of a metal including, but not limited to, stainless steel, lead, iron, molybdenum, silver, gold, titanium, aluminum, tin, or alloys of these, or laminates (layered materials) made with such metals (e.g., metal-coated ceramic, metal-coated polymer, metal-coated composite, multilayered metal materials). In some embodiments, the beam stop can be cooled, for example, with a cooling fluid such as an aqueous solution or a gas. The beam stop can be partially or completely hollow, for example, with cavities. Interior spaces of the beam stop can be used for cooling fluids and gases. The beam stop can be of any shape, including flat, curved, round, oval, square, rectangular, beveled and wedged shapes.

In some embodiments, the beam stop can have perforations so as to allow some electrons through, thus controlling (e.g., reducing) the levels of radiation across the whole area of the window, or in specific regions of the window. The beam stop can be a mesh formed, for example, from fibers or wires. Multiple beam stops can be used, together or independently, to control the irradiation. The beam stop can be remotely controlled, e.g., by radio signal or hard wired to a motor for moving the beam into or out of position.

In some embodiments, the embodiments disclosed herein can also include a beam dump when utilizing a radiation treatment. A beam dump's purpose is to safely absorb a beam of charged particles. Like a beam stop, a beam dump can be used to block the beam of charged particles. However, a beam dump is much more robust than a beam stop, and is intended to block the full power of the electron beam for an extended period of time. They are often used to block the beam as the accelerator is powering up. Beam dumps are also designed to accommodate the heat generated by such beams, and are usually made from materials such as copper, aluminum, carbon, beryllium, tungsten, or mercury. Beam dumps can be cooled, for example, using a cooling fluid that can be in thermal contact with the beam dump.

In some embodiments, various conveying systems can be used to convey the feedstock materials, for example, to a vault and under an electron beam in a vault. Exemplary conveyors are belt conveyors, pneumatic conveyors, screw conveyors, carts, trains, trains or carts on rails, elevators, front loaders, backhoes, cranes, various scrapers and shovels, trucks, and throwing devices can be used. For example, vibratory conveyors can be used in various processes described herein, for example, as disclosed in International App. No. PCT/US2013/064332, the entire disclosure of which is herein incorporated by reference.

### Chemical Treatment of Biomass

In some embodiments, or in addition, the biomass material can be treated with another treatment, for example, chemical treatments, such as with an acid (HCl, H₂SO₄, H₃PO₄), a base (e.g., KOH and NaOH), a chemical oxidant (e.g., peroxides, chlorates, ozone), irradiation, steam explosion, pyrolysis, sonication, oxidation, chemical treatment. The treatments can be in any order and in any sequence and combinations. For example, the feedstock material can first be physically treated by one or more treatment methods, e.g., chemical treatment including and in combination with acid hydrolysis (e.g., utilizing HCl, H₂SO₄, H₃PO₄), radiation, sonication, oxidation, pyrolysis or steam explosion, and then mechanically treated. This sequence can be advantageous since materials treated by one or more of the other treatments, e.g., irradiation or pyrolysis, tend to be more brittle and, therefore, it may be easier to further change the structure of the material by chemical treatment.

In some embodiments, chemical treatment can remove some or all of the lignin (for example, chemical pulping) and can partially or completely hydrolyze the material. In some embodiments, the methods described herein also can be used with prehydrolyzed material. In some embodiments, the methods described herein also can be used with material that has not been prehydrolyzed. In some embodiments, the methods can be used with mixtures of hydrolyzed and non-hydrolyzed materials, for example, with about 50% or more non-hydrolyzed material, with about 60% or more non- hydrolyzed material, with about 70% or more non-hydrolyzed material, with about 80% or more non-hydrolyzed material or even with 90% or more non-hydrolyzed material.

### Products

Using the methods described herein, a starting biomass material (e.g., plant biomass, animal biomass, paper, and municipal waste biomass) can be used as feedstock to produce useful intermediates and products such as carbohydrates, alcohols, and organic acids, (e.g., lactic acid). As described previously, in order to convert the feedstock to a form that can be readily processed, in some embodiments, the glucan- or xylan-containing cellulose in the biomass can be hydrolyzed to low molecular weight carbohydrates through a process referred to as saccharification. In some embodiments, the low molecular weight carbohydrates can then be used, for example, in an existing manufacturing plant, such as a single cell protein plant, an enzyme manufacturing plant, or a fuel plant, e.g., an ethanol manufacturing facility.

In some embodiments, the spent biomass (e.g., spent lignocellulosic material) from lignocellulosic processing by the methods described herein are expected to have a high lignin content and in addition to being useful for producing energy through combustion in a co-generation plant, may have uses as other valuable products. In some embodiments, the spent biomass can be a byproduct from the process of producing organic acids (e.g., polyhydroxy acids, alpha hydroxy acids, beta-hydroxy acids). In some embodiments, the lignin can be used as captured as a plastic, or it can be synthetically upgraded to other plastics. In some instances, it can also be converted to lignosulfonates, which can be utilized as binders, dispersants, emulsifiers or as sequestrants.

In some embodiments, when used as a binder, the lignin or a lignosulfonate can, e.g., be utilized in coal briquettes, in ceramics, for binding carbon black, for binding fertilizers and herbicides, as a dust suppressant, in the making of plywood and particle board, for binding animal feeds, as a binder for fiberglass, as a binder in linoleum paste and as a soil stabilizer. As a dispersant, the lignin or lignosulfonates can be used, e.g., concrete mixes, clay and ceramics, dyes and pigments, leather tanning and in gypsum board. As an emulsifier, the lignin or lignosulfonates can be used, e.g., in asphalt, pigments and dyes, pesticides and wax emulsions. As a sequestrant, the lignin or lignosulfonates can be used, e.g., in micro-nutrient systems, cleaning compounds and water treatment systems, e.g., for boiler and cooling systems.

In some embodiments, the lignin produced may be converted to a biofuel. For energy production lignin generally has a higher energy content than holocellulose (cellulose and hemicellulose) since it contains more carbon than homocellulose. For example, dry lignin can have an energy content of between about 11,000 and 12,500 BTU per pound, compared to 7,000 an 8,000 BTU per pound of holocellulose. As such, lignin can be densified and converted into briquettes and pellets for burning. For example, the lignin can be converted into pellets by any method described herein. For a slower burning pellet or briquette, the lignin can be crosslinked, such as applying a radiation dose of between about 0.5 Mrad and 5 Mrad. Crosslinking can make a slower burning form factor. The form factor, such as a pellet or briquette, can be converted to a “synthetic coal” or charcoal by pyrolyzing in the absence of air, e.g., at between 400 and 950° C. Prior to pyrolyzing, it can be desirable to crosslink the lignin to maintain structural integrity.

In some embodiments, lignin derived products can also be combined with poly hydroxycarboxylic acid and poly hydroxycarboxylic acid derived products. (e.g., poly hydroxycarboxylic acid that has been produced as described herein). For example, lignin and lignin derived products can be blended, grafted to or otherwise combined and/or mixed with poly hydroxycarboxylic acid. The lignin can, for example, be useful for strengthening, plasticizing or otherwise modifying the poly hydroxycarboxylic acid

Using the processes described herein, the biomass material can be converted to one or more products, such as energy, fuels, foods and materials. Specific examples of products include, but are not limited to, hydrogen, sugars (e.g., glucose, xylose, arabinose, mannose, galactose, fructose, disaccharides, oligosaccharides and polysaccharides), alcohols (e.g., monohydric alcohols or dihydric alcohols, such as ethanol, n-propanol, isobutanol, sec-butanol, tert-butanol or n-butanol), hydrated or hydrous alcohols (e.g., containing greater than 10%, 20%, 30% or even greater than 40% water), biodiesel, organic acids, hydrocarbons (e.g., methane, ethane, propane, isobutene, pentane, n-hexane, biodiesel, bio-gasoline and mixtures thereof), co-products (e.g., proteins, such as cellulolytic proteins (enzymes) or single cell proteins), and mixtures of any of these in any combination or relative concentration, and optionally in combination with any additives (e.g., fuel additives). Other examples include carboxylic acids, salts of a carboxylic acid, a mixture of carboxylic acids and salts of carboxylic acids and esters of carboxylic acids (e.g., methyl, ethyl and n-propyl esters), ketones (e.g., acetone), aldehydes (e.g., acetaldehyde), alpha and beta unsaturated acids (e.g., acrylic acid) and olefins (e.g., ethylene). Other alcohols and alcohol derivatives include propanol, propylene glycol, 1,4-butanediol, 1,3-propanediol, sugar alcohols and polyols (e.g., glycol, glycerol, erythritol, threitol, arabitol, xylitol, ribitol, mannitol, sorbitol, galactitol, iditol, inositol, volemitol, isomalt, maltitol, lactitol, maltotriitol, maltotetraitol, and polyglycitol and other polyols), and methyl or ethyl esters of any of these alcohols. Other products include methyl acrylate, methyl methacrylate, lactic acid, citric acid, formic acid, acetic acid, propionic acid, butyric acid, succinic acid, valeric acid, caproic acid, 3-hydroxypropionic acid, palmitic acid, stearic acid, oxalic acid, malonic acid, glutaric acid, oleic acid, linoleic acid, glycolic acid, gamma-hydroxybutyric acid, and mixtures thereof, salts of any of these acids, mixtures of any of the acids and their respective salts.

In some embodiments, any combination of the above products with each other, and/or of the above products with other products, which other products may be made by the processes described herein or otherwise, may be packaged together and sold as products. The products may be combined, e.g., mixed, blended or co-dissolved, or may simply be packaged or sold together.

In some embodiments, any of the products or combinations of products described herein may be sanitized or sterilized prior to selling the products, e.g., after purification or isolation or even after packaging, to neutralize one or more potentially undesirable contaminants that could be present in the product(s). Such sanitation can be done with electron bombardment, for example, be at a dosage of less than about 20 Mrad, e.g., from about 0.1 to 15 Mrad, from about 0.5 to 7 Mrad, or from about 1 to 3 Mrad.

In some embodiments, the processes described herein can produce various by-product streams useful for generating steam and electricity to be used in other parts of the plant (co-generation) or sold on the open market. For example, steam generated from burning by-product streams can be used in a distillation process. As another example, electricity generated from burning by-product streams can be used to power electron beam generators used in pretreatment.

The by-products used to generate steam and electricity are derived from a number of sources throughout the process. For example, anaerobic digestion of wastewater can produce a biogas high in methane and a small amount of waste biomass (sludge). As another example, post-saccharification and/or post-distillate solids (e.g., unconverted lignin, cellulose, and hemicellulose remaining from the pretreatment and primary processes) can be used, e.g., burned, as a fuel.

In some embodiments, many of the products obtained, such as ethanol or n-butanol, can be utilized as a fuel for powering cars, trucks, tractors, ships or trains, e.g., as an internal combustion fuel or as a fuel cell feedstock. Many of the products obtained can also be utilized to power aircraft, such as planes, e.g., having jet engines or helicopters. In addition, the products described herein can be utilized for electrical power generation, e.g., in a conventional steam generating plant or in a fuel cell plant.

Other intermediates and products, including food and pharmaceutical products, are described in U.S. Patent Publication No. 2010/0124583 A1, published May 20, 2010, to Medoff, the full disclosure of which is hereby incorporated by reference herein.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples specifically point out various aspects of the present invention, and are not to be construed as limiting in any way the remainder of the disclosure.

### Example 1

### Preparation of Lysed Cell Matter

Cell culture stock: Trichoderma reesei strain RUT-C30 (ATCC 56765) was used to produce the lysed cell matter. The cell culture was rehydrated and propagated in potato dextrose (PD) media at 25° C. To propagate Trichoderma reesei cells, 40 μl of rehydrated cells were used to inoculate potato dextrose agar (PDA) solid medium. Rehydrated cells were also inoculated into 50 mL of PD liquid medium and incubated at 25° C. and 200 rpm. After 2 weeks incubation in PDA media, spores were resuspended in a sterile solution of NaCl (9 g/L) and 20% glycerol, and stored at −80° C. for use as a cell bank.

Protein measurement and cellulase assay: Protein concentration was measured by the Bradford method using bovine serum albumin as a standard.

Sugar concentrations were analyzed on a YSI 7100 Multiparameter Bioanalytical System (YSI Life Sciences, Yellow Springs, Ohio, USA), while other products were analyzed by HPLC.

Media: The media for cell propagation comprises corn steep (2 g/L), ammonium sulfate (1.4 g/L), potassium hydroxide (0.8 g/L), phosphoric acid (85%, 4 mL/L), phthalic acid dipotassium salt (5 g/L), magnesium sulfate heptahydrate (0.3 g/L), calcium chloride (0.3 g/L), ferrous sulfate heptahydrate (5 mg/L), manganese sulfate monohydrate (1.6 mg/L), zinc sulfate heptahydrate (5 mg/L) and cobalt chloride hexahydrate (2 mg/L). The media is described in Herpoel-Gimbert et al., Biotechnology for Biofuels, 2008, 1:18.

Bioreactor: The freezer stock described above was used to prepare the seed culture using the media prepared as outlined with 2.5% additional glucose. The seed culture was typically grown in a flask at 30° C. and 200 rpm for 72 hrs. Seed culture broth (50 mL) was used as an inoculum in a 1 L culture carried out in a 3 L fermenter. In the growth phase, 35 g/L of lactose was added to the medium. The culture conditions were as follows: 27° C., pH 4.8 (with 6M ammonia), air flow 0.5 vessel volumes per minute (VVM), mixing at 500 rpm, and dissolved oxygen (DO) maintained above 40% oxygen saturation. The biomass was milled corn cob collected between mesh sizes of 15 and 40. Treatment of the biomass involved electron bombardment with an electron beam for a total dose of 35 Mrad. During fermentation, antifoam 204 (Sigma) was injected into the culture when the foam reached the fermenter head. The fermentation proceeded for 11 days. The culture supernatant was harvested by centrifugation at 14,500 rpm for 5 minutes and stored at 4° C. The precipitate was blended for 30-60 seconds to lyse the fungal cell matter, and the lysed material was stored at 4° C.

### Example 2

### Determination of Fermentation Composition Components

In order to determine the optimal conditions for production of products of the current invention (e.g., organic acids (e.g., lactic acid)), fermentation reactions were conducted on a small test scale. Sterile lactobacillus MRS broth (Difco™, 100 mL) was inoculated with 1% L. rhamnosus (NRRL B-445,for L-lactic acid) or L. coryniformis (NRRL B-4390, for D-lactic acid) and grown overnight. Each flask contained varying amounts of lysed cell matter, yeast extract, and/or other additive, 6 wt. % CaCO₃, and inoculum. The pH of each test shake flask was approximately between 6 and 7. The flasks were placed into shaker incubators at 37° C. and 125 rpm and sampled periodically.

Small scale fermentation reactions were conducted in bioreactors (1.3 L capacity) charged with 700 mL media. The reactors contained various amounts of additives as shown in Table 2 below. The reactors were heated to 70° C., and the pH of the reactors was maintained at 6.5 using 6N NaOH. After one hour, the reactors were cooled to 37° C. and the pH was adjusted again to about pH 6.5. The reactors were then inoculated with 1% ATCC 445 (for L-lactic acid) or ATCC 4390 (for D-lactic acid) and grown overnight as described above.

The additives tested are summarized in Table 2 and include: 1) lysed cell matter from Trichoderma 2) yeast extract (Fluka), and 3) chitin powder (Alfa Aesar). Concentrations are given in g/L. Preparation of the lysed Trichoderma cell matter is outlined in Example 1. The sugars (e.g., glucose, xylose) were isolated from saccharification of biomass, which was pre-treated to 35 Mrad with electrons from an electron beam. The pre-treated biomass was added to water to produce a 35% by weight slurry. Sulfuric acid was added to the slurry until the concentration of sulfuric acid was 0.1% by weight and the pH was approximately 4.0. The acidified slurry was heated to 140° C. for 30 min. The slurry was cooled to 48-50° C. and saccharified by adding enzyme (1.2 g/L) and stirring with a jet mixer for 3 days.

Table 2 summarizes results for various combinations of additives used in a fermentation reaction wherein the fermentation agent was L. rhamnosus strain B-445 obtained from NRRL. After inoculation, the flasks were held at 37° C. for 48 hours and then sampled.

TABLE 2
Summary of fermentation composition components and products

| | | | Lactic | | |
|---|---|---|---|---|---|
| Reaction | Additive | Additive | Acid, | Glucose, | Xylose, |
| Number | One | Two | g/L | g/L | g/L |
| 1 | 2 g/L Yeast | 5 g/L Chitin | N.D. | 51 | 51 |
| | Extract | | | | |
| 2 | 2 g/L Yeast | 10 g/L Chitin | N.D. | 52 | 49 |
| | Extract | | | | |
| 3 | 2 g/L Yeast | 50 g/L Chitin | N.D. | 51 | 48 |
| | Extract | | | | |
| 4 | 2 g/L Yeast | | N.D. | 35 | 31 |
| (Control) | Extract | | | | |
| 5 | 2 g/L Yeast | {Diluted with | N.D. | 52 | 51 |
| (Control) | Extract | 50% water} | | | |
| 6 | 50% of | | 36 | N.D. | 31 |
| | liquid from | | | | |
| | Trichoderma | | | | |
| | cell matter | | | | |

N.D. Not Detected

As summarized in Table 2, only the fermentation composition comprising the lysed Trichoderma cell matter facilitated the conversion of the glucose to lactic acid (Reaction No. 6). Chitin did not facilitate the conversion of glucose and xylose to lactic acid at the three different levels tested.

### Example 3

### Determination of Optimal Lysed Cell Matter Concentration

Using the bioreactor procedure described in Example 2, three different concentrations of lysed cell matter were tested in the fermentation reaction. Glucose was isolated from a saccharification batch similar to that outlined in Example 2, and the lysed cell matter was prepared as described in Example 1. The fermentation agent was L. rhamnosus strain B-445 obtained from NRRL. Samples were removed from the reactor periodically to analyze the reaction progress, e.g., amount of unreacted sugars and L-lactic acid produced. Table 3 summarizes the effect of various concentrations of lysed cell matter on the production of L-lactic acid.

TABLE 3
Effect of lysed cell matter at different concentrations.
Reaction 1:
50% lysed fungal cell matter/50% aqueous saccharified biomass

| | | | | | | |
|---|---|---|---|---|---|---|
| Time, hours | 0 | 18 | 24 | 42 | 48 | 66 |
| Glucose, g/L | 33 | 15 | 3 | 0 | 0 | 0 |
| L-Lactic acid, g/L | 0 | 16 | 28.5 | 34 | 35 | 37 |

Reaction 2:
20% lysed fungal cell matter/80% aqueous saccharified biomass

| | | | | | | |
|---|---|---|---|---|---|---|
| Time, hours | 0 | 18 | 24 | 42 | 48 | 66 |
| Glucose, g/L | 45 | 41 | 35 | 20 | 17.5 | 9 |
| L-Lactic acid, g/L | 0 | 2.5 | 7.5 | 19 | 22.5 | 29 |

Reaction 3:
10% lysed fungal cell matter/90% aqueous saccharified biomass

| | | | | | | |
|---|---|---|---|---|---|---|
| Time, hours | 0 | 18 | 24 | 42 | 48 | 66 |
| Glucose, g/L | 50 | 48 | 46 | 39 | 38 | 32 |
| L-Lactic acid, g/L | 0 | 0 | 1.5 | 8 | 9 | 13 |

As described in Table 3, a concentration of 50% lysed fungal cell matter is sufficient to provide 100% conversion of glucose to L-lactic acid in ˜40 hours. At a concentration of 20% lysed fungal cell matter, the rate of formation of lactic acid observed is slower at ˜40 hours (about 55% conversion), and is even slower when 10% lysed fungal cell matter is used in the reaction (22% conversion at ˜40 hours).

### Example 4

### Use of Aqueous Products Derived from Saccharified Biomass as the Diluent in Fermenation

Using the bioreactor procedure described above in Examples 2 and 3, the lysed fungal cell matter was diluted with the aqueous products isolated from saccharified biomass in the place of water as outlined in Example 3 (Reactions 1-3). The aqueous products were isolated from a saccharification batch similar to that described in Example 2. The lysed cell matter was prepared as described in Example 1, and the fermentation reaction was set up as described in Examples 2 and 3 using L. rhamnosus (NRRL B-445) as the fermentation agent. Samples were taken periodically to analyze for the presence of unreacted sugars and L-lactic acid formation.

TABLE 4
Effective of dilution of the fermentation
reaction with aqueous products
derived from saccharified biomass

| Time, hours | 0 | 18 | 24 |
|---|---|---|---|
| Glucose, g/L | 39 | 13.3 | 0.85 |
| Xylose, g/L | 37 | 36 | 35 |
| L-Lactic acid, g/L | 0 | 24 | 37.5 |

As detailed in Table 4, the dilution of the fermentation reaction with the aqueous products of saccharification in the presence of lysed cell matter resulted in excellent conversion of glucose to L-lactic acid.

### Example 5

### Effect of Lysed Cell Matter on Stereoisomer Ratios

The bioreactor procedure described above in Examples 2 and 3 was carried out to evaluate the effect of lysed cell matter on stereoisomer ratios. The aqueous products were isolated from a saccharification batch in a similar manner to that described in Examples 2 and 3, and the lysed cell matter was prepared as detailed in Example 1, except that the lysis was carried out with glass beads. The fermentation agent was L. rhamnosus B-445 obtained from NRRL. The fermentation resulted in a yield of 22.76 g/L lactic acid with an L:D ratio of 94.4:5.64 or 16.7:1

### Example 6

### Effect of Lysed Cell Matter on Stereoisomer Ratios

The bioreactor procedure described above in Examples 2 and 3 was done carried out to evaluate the effect of lysed cell matter on stereoisomer ratios. The aqueous products were isolated from a saccharification batch in a similar manner to that described in Examples 2 and 3, and the lysed cell matter was prepared as detailed in Example 1, except that the lysis was carried out with glass beads. The fermentation agent was L. coryniformis (B-4390) obtained from NRRL. The fermentation resulted in a yield of 13.7 g/L lactic acid with an L:D ratio of 1.46:98.6 or 1:67.5.

### Example 7

### Comparison of Methods of Cell Disruption

The bioreactor procedure described above in Examples 2 and 3 was carried out to compare different preparation methods of the lysed cell matter. The aqueous products were isolated from a saccharification batch in a similar manner to that described in Examples 2 and 3, followed by mixture of the aqueous saccharification products with the lysed cell matter. The mixture was then blended and centrifuged. In the first reaction, only the supernatant was used in fermentation. In the second reaction, the lysed cell matter was suspended in water and then added in a 1:1 ratio to the aqueous products of the saccharification step.

### Example 8

### Effect of Alternate Fermentation Agents

A starter culture of Actinobacillus succinogenes (ATCC 55618) in tryptic soy broth (TSB, Bacto 257107) was inoculated with 0.1% inoculum from a freezer stock. The culture was incubated at 30° C. with shaking at 125 rpm for 24 hours. Fermentation media contained NaH₂PO₄ (1.72 g/L), Na₂HPO₄ 7H₂O (2.84 g/L), NaCl (1 g/L), MgCl₂ (0.2 g/L), CaCl₂ (0.2 g/L), as well as a 1:1 ratio of Trichoderma extract (prepared as described in Example 1) and the aqueous products from the saccharification reaction, as well as additional components detailed in Table 5 below.

The media was heated to 70° C. for 1 hour and then cooled to 37° C. while agitated at 200 rpm and sparged throughout with 100 ccm CO₂. The pH of the media was adjusted to 7.0 using 6N NaOH, and inoculated with 1% starter culture. The pH of the media was then maintained at 7.0 for the duration of fermentation with 6N NaOH. Over the course of the next three days, the media was sampled for the conversion of sugars to succinic acid and lactic acid. A summary of conversion ratios in various reaction conditions is presented in Table 5.

TABLE 5
Effect of fermentation conditions on organic acid formation.

| Time | | Succinic Acid | Glucose | Xylose | Lactic Acid | Acetic Acid |
|---|---|---|---|---|---|---|
| (Hr) | Conditions | (g/L) | (g/L) | (g/L) | (g/L) | (g/L) |
| 0 | 618 HH w/50% Trichoderma | | 33.125 | 32.934 | | 5.127 |
| 0 | 618 HH w/50% Trichoderma | | 32.581 | 32.351 | | 5.061 |
| 0 | 618 ED xylose w/50% Trichoderma | | 5.517 | 12.659 | 0.725 | 1.083 |
| 0 | 618 ED xylose w/50% Trichoderma | | 5.531 | 12.591 | 0.735 | 1.173 |
| 0 | 618 ED xylose w/20 g/L YE | | | 7.604 | 0.729 | |
| 0 | 618 ED xylose w/20 g/L YE | | | 8.194 | 0.778 | |
| 18 | 618 HH w/50% Trichoderma | | 31.473 | 31.177 | | 5.611 |
| 18 | 618 HH w/50% Trichoderma | | 31.409 | 31.119 | | 5.582 |
| 18 | 618 ED xylose w/50% Trichoderma | 13.624 | | | 0.659 | 6.161 |
| 18 | 618 ED xylose w/50% Trichoderma | 13.692 | | | 0.747 | 6.477 |
| 18 | 618 ED xylose w/20 g/L YE | 6.04 | | | 0.628 | 3.748 |
| 18 | 618 ED xylose w/20 g/L YE | 6.133 | | | 0.614 | 3.712 |
| 24 | 618 HH w/50% Trichoderma | | 31.292 | 31.008 | | 5.877 |
| 24 | 618 HH w/50% Trichoderma | | 30.419 | 30.526 | | 6.02 |
| 24 | 618 ED xylose w 50% Trichoderma | 13.971 | | | 0.695 | 6.461 |
| 24 | 618 ED xylose w/50% Trichoderma | 13.818 | | | 0.743 | 6.817 |
| 24 | 618 ED xylose w/20 g/L YE | 6.233 | | | 0.617 | 4.069 |
| 24 | 618 ED xylose w/20 g/L YE | 6.386 | | | 0.617 | 4.227 |
| 42 | 618 HH w/50% Trichoderma | 3.199 | | 25.374 | 25.364 | 6.407 |
| 42 | 618 HH w/50% Trichoderma | 10.658 | | 24.874 | 2.565 | 8.071 |
| 42 | 618 ED xylose w/50% Trichoderma | 14.343 | | | 1.157 | 6.797 |
| 42 | 618 ED xylose w/50% Trichoderma | 14.104 | | | 1.122 | 6.839 |
| 42 | 618 ED xylose w/20 g/L YE | 6.317 | | | 0.589 | 4.547 |
| 42 | 618 ED xylose w/20 g/L YE | 6.268 | | | 0.793 | 4.729 |

### Equivalents

The disclosures of each and every patent, patent application, and publication cited herein are hereby incorporated herein by reference in their entirety. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference.

While this invention has been particularly shown and described with references to preferred embodiments thereof, it will be understood by those skilled in the art that various changes in form and details may be made therein without departing from the scope of the invention encompassed by the appended claims.

1. A method of making a product, the method comprising contacting one or more sugars with a fermentation composition comprising lysed cell matter to produce the product.

2. The method of claim 1, wherein the one or more sugars comprise xylose and glucose.

3. The method of claim 1, wherein the one or more sugars are formed by saccharifying a biomass material comprising lignocellulosic material.

4. The method of claim 3, wherein the lignocellulosic material comprises an agricultural product or waste, a paper product or waste, a forestry product or waste, or a general waste.

5-8. (canceled)

9. The method of claim 3, wherein the lignocellulosic material has been pretreated to reduce its recalcitrance by treating the lignocellulosic material with an electron beam, sonication, oxidation, pyrolysis, steam explosion, heat treatment, chemical treatment, mechanical treatment, or freeze grinding.

10-11. (canceled)

12. The method of claim 1, wherein the one or more sugars are isolated prior to contact with the fermentation composition.

13. The method of claim 1, wherein the lysed cell matter comprises lysed fungal cells.

14. The method of claim 13, wherein the fungal cells comprise a species in the genera selected from Coprinus, Myceliophthora, Scytalidium, Penicillium, Aspergillus, Humicola, Fusarium, Thielavia, Acremonium, Chrysosporium, Saccharomyces, Candida, Clavispora, Pichia, Yarrowia, and Trichoderma.

15. (canceled)

16. The method of claim 15, wherein the fungal cells comprise the species Trichoderma reesei.

17. The method of claim 16, wherein the Trichoderma reesei comprises any individual strain, variant, or mutant thereof.

18-19. (canceled)

20. The method of claim 1, wherein the concentration of lysed cell matter in the fermentation composition is greater than or equal to 1%.

21-22. (canceled)

23. The method of claim 1, wherein the fermentation composition further comprises a fermentation agent.

24-26. (canceled)

27. The method of claim 23, wherein the fermentation agent comprises the one or more bacteria in the genera selected from Bacillus, Actinobacillus, Lactobacillus, Leuconostoc, Pediococcus, Lactococcus, Streptococcus, Weisella, and Pseudomonas.

28. The method of claim 1, wherein the fermentation composition further comprises an additive.

29. The method of claim 28, wherein the additive comprises a surfactant, an antifoaming agent, an antimicrobial agent, a pH adjusting agent, a solid support, or a processed cell product.

30-36. (canceled)

37. The method of claim 1, wherein the product comprises lactic acid.

38-47. (canceled)

48. A composition comprising one or more sugars and a fermentation composition comprising lysed cell matter.

49. The composition of claim 48, wherein the one or more sugars comprise xylose and glucose.

50-53. (canceled)

54. The composition of claim 48, wherein the lysed cell matter comprises lysed fungal cells.

55-56. (canceled)

57. The composition of claim 54, wherein the fungal cells comprise the species Trichoderma reesei.

58. The composition of claim 57, wherein the Trichoderma reesei comprises any individual strain, variant, or mutant thereof.

59-60. (canceled)

61. The composition of claim 48, wherein the concentration of lysed cell matter in the fermentation composition is greater than or equal to 1%.

62-63. (canceled)

64. The composition of claim 48, wherein the fermentation composition further comprises a fermentation agent.

65-67. (canceled)

68. The composition of claim 67, wherein the fermentation agent comprises one or more bacteria in the genera selected from Bacillus, Actinobacillus, Lactobacillus, Leuconostoc, Pediococcus, Lactococcus, Streptococcus, Weisella, and Pseudomonas.

69. The composition of claim 48, wherein the fermentation composition further comprises an additive.

70-71. (canceled)

72. A composition comprising a product produced by contacting one or more sugars with a fermentation composition comprising lysed cell matter.

73. The composition of claim 72, wherein the product comprises carbohydrates, alcohols, and organic acids.

74-75. (canceled)

76. The composition of claim 72, wherein the product comprises lactic acid.

77. The composition of claim 76, wherein the product comprises nearly pure L-lactic acid or nearly pure D-lactic acid.

78. The composition of claim 76, wherein the product comprises a mixture of L-lactic acid and D-lactic acid.

79. The composition of claim 78, wherein the mixture comprises a ratio of L-lactic acid to D-lactic acid of about 60:40.

80. The composition of claim 78, wherein the mixture comprises a ratio of L-lactic acid to D-lactic acid of about 80:20.

81. The composition of claim 78, wherein the mixture comprises a ratio of L-lactic acid to D-lactic acid of about 90:10.

82. The composition of claim 78, wherein the mixture comprises a ratio of L-lactic acid to D-lactic acid of about 95:5.
